(19) United States
(12) Patent Application Publication
(10) Pub. No.: US 2026/0363645 A1
(43) Pub. Date: Nov. 19, 2026

(54) SURGERY ASSISTANCE DEVICE, METHOD OF ASSISTING SURGERY, AND COMPUTER PROGRAM

(71) Applicant: ASAHI INTECC CO., LTD., Seto-shi (JP)

(72) Inventor: Koki KATO, Seto-shi (JP)

(73) Assignee: ASAHI INTECC CO., LTD., Seto-shi (JP)

(21) Appl. No.: 19/571,909

(22) Filed: Mar. 19, 2026

(30) Foreign Application Priority Data
Mar. 19, 2025 (JP) 2025-044708

(51) Int. Cl. A61B 6/00; A61B 6/42; A61B 6/50; A61B 90/00; G06T 11/60

(52) U.S. Cl. CPC A61B 6/5294; A61B 6/4233; A61B 6/4452; A61B 6/504; A61B 6/547; G06T 11/60; A61B 2090/3764; G06T 2210/41

(57) ABSTRACT

A surgery assistance device includes an image generation unit that generates an image showing a placement position of a flat panel detector (FPD) when a blood vessel is imaged using the FPD, which can be moved along the surface of a virtual sphere centered at the origin of a three-dimensional space. The image generation unit acquires range of motion information that defines where the FPD can be moved, and generates a two-dimensional image showing a placement position of the FPD in two-dimensional coordinates, and which is an image including a planar trajectory on which the placement position of the FPD is located in a direction perpendicular to an extending direction of the blood vessel, and a recommended range showing a range derived using the range of motion information and planar trajectory, and which indicates a range that is recommended as the placement position of the FPD.

### CROSS-REFERENCE TO RELATED APPLICATIONS

The present application claims the benefit of Japanese Patent Application No. 2025-044708 filed on Mar. 19, 2025, the disclosure of which is incorporated by reference in its entirety.

### TECHNICAL FIELD

The present disclosure relates to a surgery assistance device, a method of assisting a surgery, and a computer program.

### BACKGROUND ART

In recent years, flat panel detectors are being used in blood vessel imaging that is carried out in examinations and therapy. Hereinafter, a flat panel detector is also referred to as an “FPD”. FPD stands for flat panel detector. An FPD is a device that obtains images by acquiring X-rays that have been transmitted through a human body, and then converting the X-rays into a digital signal. An FPD is also referred to as a flat panel X-ray detector. Such FPDs have the advantages of being able to achieve higher-definition image quality, shorter image display times, and lower radiation exposure compared to conventional CR systems. CR stands for computed radiography. An imaging device including an FPD is also referred to as a radiation imaging device. For example, JP 2013-233413 A discloses, in a radiation imaging device, the generation of a graph that includes points indicating the recommended angle of a C-arm to which an FPD is attached, and a movable range of the C-arm.

### SUMMARY

### Technical Problem

An operator acquires an image of a blood vessel subjected to examination or therapy using an FPD, and operates a medical device while confirming the positions of each of the blood vessel, a lesion, and the medical device that has been inserted into the blood vessel included in the image. The blood vessel subjected to examination or therapy is referred to as a “target blood vessel” below. For this reason, in a case where the placement position of the FPD is not appropriate, there is a problem that the operator is unable to correctly grasp the positional relationship between the target blood vessel, the lesion, and the medical device, and the medical device cannot be pushed forward in an intended direction. The medical device is a guide wire or catheter. In this respect, because the technique described in JP 2013-233413 A merely displas a graph that includes a point indicating the recommended angle of the C-arm, and a movable range of the C-arm, in a case where the points displayed on the graph are located outside the movable range, there was a problem that the surgeon (operator) had difficulty with the subsequent procedures.

### Solution to Problem

The present disclosure has been made to solve at least part of the problem described above, and can be implemented as the following aspect.

According to an aspect of the present disclosure, a surgery assistance device is provided. The surgery assistance device includes circuitry that generates an image showing a placement position of a flat panel detector (FPD) when a target blood vessel is imaged using the FPD, which can be moved along a surface of a virtual sphere having a predetermined radius and being centered at an origin of a three-dimensional space. The circuitry acquires range of motion information that defines a first range in which the FPD can be moved, and generates a two-dimensional image showing a placement position of the FPD in two-dimensional coordinates, and which is a two-dimensional image including a first planar trajectory on which the placement position of the FPD is located in a first direction, which is perpendicular to an extending direction of the target blood vessel, and a recommended range showing a range derived using the range of motion information and the first planar trajectory, and which indicates a range that is recommended as the placement position of the FPD.

### BRIEF DESCRIPTION OF DRAWINGS

FIG. 1 is an explanatory diagram showing an example of a surgery assistance system.

FIG. 2 is a diagram that illustrates an LAO.

FIG. 3 is a diagram that illustrates an RAO.

FIG. 4 is a diagram that illustrates a CRA.

FIG. 5 is a diagram that illustrates a CAU.

FIG. 6 is a flowchart showing an example of the processing procedure of guide processing.

FIG. 7 is a diagram showing an example of a first screen in an initial state.

FIG. 8 is an explanatory diagram of an acquisition method of a target blood vessel using the first screen.

FIG. 9 is a diagram showing a generation method of a first heat map.

FIG. 10 is a diagram showing an example of a first heat map.

FIG. 11 is a diagram showing an example of a third screen.

FIG. 12 is a diagram showing a generation method of a second heat map.

FIGS. 13A-13D is a diagram showing a generation method of a second heat map.

FIG. 14 is a diagram showing an example of a second heat map.

FIG. 15 is a diagram describing the summation of a first heat map and a second heat map.

FIG. 16 is a diagram showing an example of a composite image.

FIG. 17 is a diagram showing another example of a composite image.

FIG. 18 is a diagram showing another example of a composite image.

FIGS. 19A-19B is a diagram describing a calculation method of a recommended position in step S44 of FIG. 6.

FIG. 20 is an explanatory diagram showing an example of a surgery assistance system according to a second embodiment.

FIG. 21 is a diagram showing an example of a two-dimensional image according to the second embodiment.

FIG. 22 is a diagram showing an example of a two-dimensional image according to a third embodiment.

FIG. 23 is a diagram showing an example of a composite image according to a fourth embodiment.

FIG. 24 is a flowchart showing an example of the processing procedure of guide processing according to a fifth embodiment.

FIG. 25 is a diagram showing an example of a composite image according to the fifth embodiment.

FIG. 26 is a flowchart showing an example of the processing procedure of guide processing according to a sixth embodiment.

FIG. 27 is a diagram showing an example of a composite image according to the sixth embodiment.

FIG. 28 is a diagram showing an example of a third screen according to a seventh embodiment.

FIG. 29 is a flowchart showing an example of the processing procedure of guide processing according to an eighth embodiment.

FIG. 30 is a diagram showing an example of a composite image according to the eighth embodiment.

FIG. 31 is an explanatory diagram showing an example of a surgery assistance system according to a ninth embodiment.

FIG. 32 is a diagram showing an example of a composite image according to a tenth embodiment.

FIG. 33 is a diagram showing an example of a composite image according to an eleventh embodiment.

### DESCRIPTION OF EMBODIMENTS

### First Embodiment

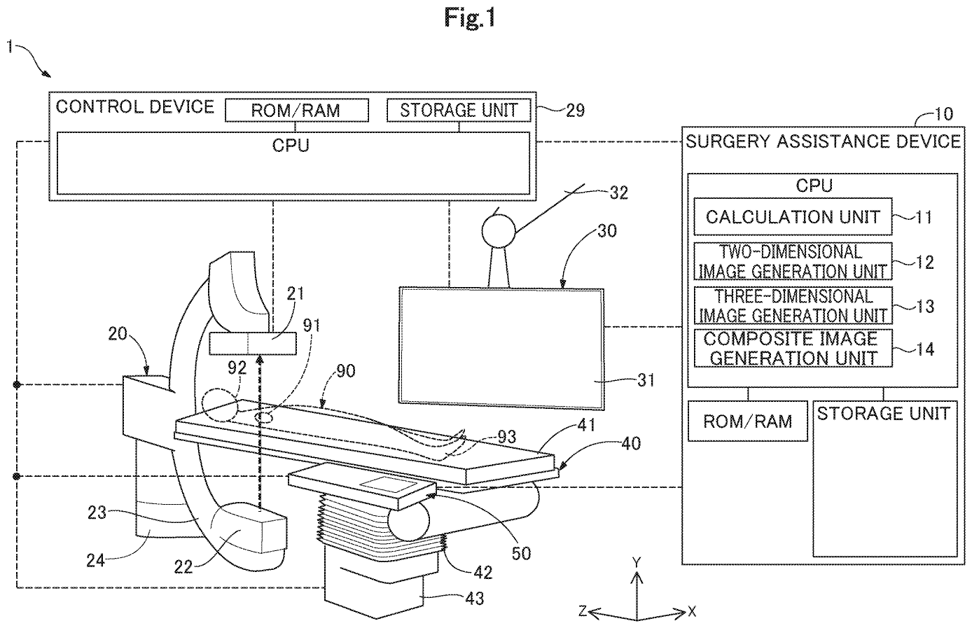

FIG. 1 is an explanatory diagram showing an example of a surgery assistance system 1. The surgery assistance system 1 is a system that acquires an X-ray image of a human body for examination or therapy, and is also referred to as a “blood vessel imaging system”. The surgery assistance system 1 includes a surgery assistance device 10, a radiation imaging device 20 having a flat panel detector, a display device 30, a table 40, and an operation unit 50. Hereinafter, a flat panel detector is also referred to as an “FPD”. FPD stands for flat panel detector. The surgery assistance system 1 according to the present embodiment is capable of guiding the range of placement positions of the FPD as a result of including the surgery assistance device 10 described below. The “target blood vessel” refers to the blood vessel subjected to examination or therapy. The surgery assistance system 1 may be used not only in the blood vessel system, but also in biological lumens such as the lymphatic system, biliary system, urinary system, respiratory system, digestive system, secretory glands, and reproductive organs.

FIG. 1 illustrates mutually orthogonal X, Y, and Z axes. The X-axis corresponds to the width direction of the radiation imaging device 20. The Y-axis corresponds to the height direction of the radiation imaging device 20. The Z-axis corresponds to the depth direction of the radiation imaging device 20. In the following description, the direction in which the head 92 of a human body 90 having the target blood vessel is also referred to as the “Z-axis direction”, and is sometimes represented as “Z”. The human body 90 represents a patient.

The surgery assistance device 10 generates, as a result of the guide processing described below, an image representing a range of placement positions of the FPD of the radiation imaging device 20, and outputs the generated image to the display device 30. The surgery assistance device 10 includes a CPU, a ROM, a RAM, and a storage unit. The surgery assistance device 10, as a result of the CPU executing a computer program that is stored in the ROM, realizes the functions of a calculation unit 11, a two-dimensional image generation unit 12, a three-dimensional image generation unit 13, and a composite image generation unit 14. The surgery assistance device 10 is electrically connected to each of a control device 29 of the radiation imaging device 20, the display device 30, and the operation unit 50. The surgery assistance device 10 transmits information to, and receives information from, the control device 29 of the radiation imaging device 20, the display device 30, and the operation unit 50.

The functions realized by the constituent elements described herein may be implemented by circuitry or processing circuitry including a general-purpose processor, an application-specific processor, an integrated circuit, an application-specific integrated circuit (ASIC), a central processing unit (CPU), a conventional circuit, and/or any combination thereof, which have been programmed to realize the described functions. The processor includes transistors and other circuits, and is regarded as circuitry or processing circuitry. The processor may be a programmed processor which executes a program stored in a memory. Herein, circuitry, units, and means are hardware programmed to realize the described functions, or hardware that executes the described functions. The hardware may be any type of hardware disclosed herein, or any type of hardware that has been programmed to achieve the described functions or known to execute the described functions. In a case where the hardware is a processor which is regarded as circuitry, the circuitry, means, or unit is a combination of hardware and software used to configure the hardware and/or the processor.

The calculation unit 11 calculates (derives) a blood vessel axis of the target blood vessel, and first to fourth planar trajectories PO1 to PO4. The two-dimensional image generation unit 12 generates an image showing the range of placement positions of the FPD when the operator uses the FPD to image the target blood vessel. The operator is a physician that performs the procedure, or a physician's assistant. The image generated by the two-dimensional image generation unit 12 is a two-dimensional image showing the placement position of the FPD in two-dimensional coordinates. The two-dimensional image generation unit 12 is also simply referred to as an “image generation unit”. The three-dimensional image generation unit 13 generates an image representing the range of placement positions of the FPD when the operator uses the FPD to image the target blood vessel. The image generated by the three-dimensional image generation unit 13 is a three-dimensional image showing the placement position of the FPD in three-dimensional coordinates. The composite image generation unit 14 generates a composite image in which the two-dimensional image that has been generated by the two-dimensional image generation unit 12 and the three-dimensional image that has been generated by the three-dimensional image generation unit 13 have been combined, and then outputs the composite image to the display device 30. These details will be described later.

The radiation imaging device 20 includes the FPD. The radiation imaging device 20 is a device that obtains an X-ray image by acquiring X-rays that have been transmitted through a human body, and then converting the X-rays into a digital signal. The X-ray image acquired by the radiation imaging device 20 is also referred to as an “angiographic image”. The radiation imaging device 20 includes the FPD 21, an X-ray tube device 22, a C-arm 23, a support portion 24, and the control device 29.

The FPD 21 includes a flat panel X-ray detector. The FPD 21 converts the X-rays that have entered from the X-ray tube device 22 into an electrical signal, performs A/D conversion, and then generates an X-ray image. A/D refers to analog/digital. The X-ray tube device 22 receives a high voltage output from an X-ray high voltage device (not shown), and irradiates an X-ray beam. As indicated by the bold dashed line in the Y-axis direction in FIG. 1, the X-ray beam irradiated from the X-ray tube device 22 enters the FPD 21 via the human body 90. The C-arm 23 is a letter C-shaped arm that fixes the FPD 21 and the X-ray tube device 22 in opposing positions. The C-arm 23 is a support that supports the FPD 21 and the X-ray tube device 22. The support portion 24 rotatably supports the C-arm 23. That is, the FPD 21 and the X-ray tube device 22 can be moved to an arbitrary imaging position around the human body 90 lying on a bed 41 while being fixed in opposing positions by the C-arm 23. The radiation imaging device 20 is also sometimes simply referred to as an “FPD” or “FPD device”.

The control device 29 includes a CPU, a ROM, a RAM, and a storage unit. In the control device 29, the CPU controls the entire radiation imaging device 20 by executing a computer program stored in the ROM. The control device 29 is electrically connected to each of the FPD 21, the support portion 24, the display device 30, the table 40, and the operation unit 50. The control device 29 displays the X-ray image generated by the FPD 21 on the display device 30. The control device 29 drives the support portion 24 and rotates the C-arm 23 according to an operation from the operation unit 50. The control device 29 changes the height of the bed 41 by extending or retracting an extending/retracting portion 42, and changes the position of the bed 41 by moving the table 40 in the Z-axis direction according to operations from the operation unit 50.

The display device 30 is connected to the surgery assistance device 10 and the control device 29. The display device 30 is an output interface to the surgery assistance device 10 and the radiation imaging device 20. The display device 30 includes a monitor 31 and an arm 32. As the monitor 31, it is possible to adopt a known means such as a liquid-crystal display, smart glasses, or a projector. The display device 30 is also referred to as a “display unit”. The arm 32 supports and fixes the monitor 31. In a case where the monitor 31 represents smart glasses or a projector, the arm 32 may be omitted.

The table 40 is a stage for the human body 90 to lie on and be positioned near the FPD 21. The table 40 includes the bed 41, the extending/retracting portion 42, and a leg portion 43. The bed 41 includes a mattress on which the human body 90 can lay down. The bed 41 is supported by the table 40 so as to be movable in the Z-axis direction. The extending/retracting portion 42 changes the height of the bed 41 by extracting and contracting in the Y-axis direction. The leg portion 43 supports the bed 41 and the extending/retracting portion 42. As indicated by the dashed line in FIG. 1, the human body 90 is laid facing upward on the bed 41, in a state where the head 92 is placed on the side closer to the FPD 21, and the feet 93 are placed on the side further away from the FPD 21. In this way, it is easy to acquire an image of the target blood vessel in the heart 91 using the FPD 21.

The operation unit 50 is connected to the surgery assistance device 10 and the control device 29. The operation unit 50 is an input interface to the surgery assistance device 10 and the radiation imaging device 20. As the operation unit 50, a known means can be adopted, such as a touch panel, an operation button, an operation lever, an operation switch, a keyboard, a mouse, a voice input unit, or a foot switch. The operation unit 50 is also referred to as an “input unit”. In the illustrated example, the operation unit 50 is fixed to the table 40.

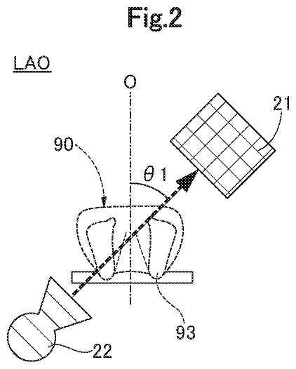

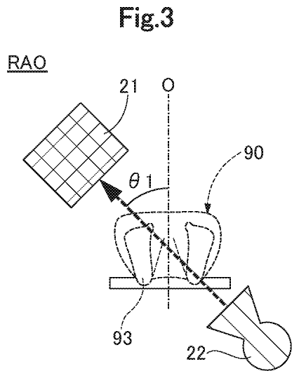

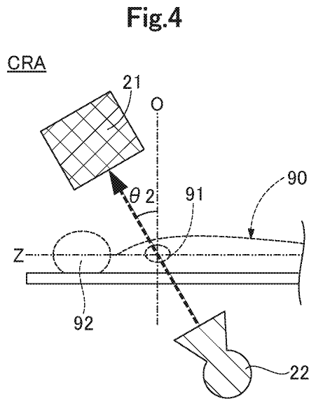

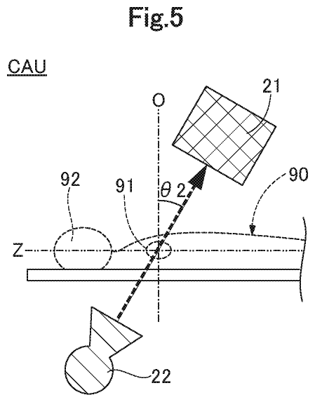

FIG. 2 is a diagram that illustrates an LAO. FIG. 3 is a diagram that illustrates an RAO. FIG. 4 is a diagram that illustrates a CRA. FIG. 5 is a diagram that illustrates a CAU. The placement position of the FPD 21 will be described using FIGS. 2 to 5. The placement position of the FPD 21 is, in other words, the placement position of the C-arm 23. As shown in FIG. 2, a case where the FPD 21 is positioned in the left direction of the human body 90 is referred to as an LAO. LAO stands for left anterior oblique view. As shown in FIG. 3, a case where the FPD 21 is positioned in the right direction of the human body 90 is referred to as an RAO. RAO stands for right anterior oblique view. As shown in FIG. 4, a case where the FPD 21 is positioned in the upper direction of the human body 90 is referred to as a CRA. CRA stands for cranial. As shown in FIG. 5, a case where the FPD 21 is positioned in the lower direction of the human body 90 is referred to as a CAU. CAU stands for caudal. That is, the “placement position of the FPD 21” is specified by a combination of a left-right position a1 and an up-down position a2 as described below:
(a1) LAO or RAO, and an angle θ1 from the center O of the human body 90; and
(a2) CRA or CAU, and an angle θ2 from the center O of the human body 90.

For example, “RAO 28 CRA 5” means that the FPD 21 is positioned at a 28 degrees angle in the right direction of the human body 90, and is positioned at a 5 degree angle in the upper direction of the human body 90.

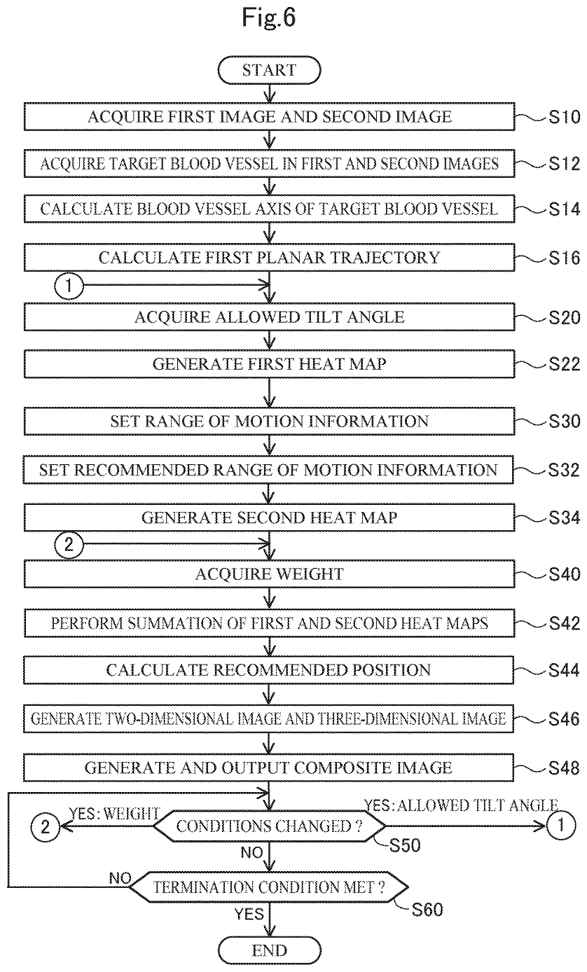

FIG. 6 is a flowchart showing an example of the processing procedure of guide processing. The guide processing can be started at any time. The guide processing can be started, for example, when a predetermined application in the surgery assistance device 10 is started.

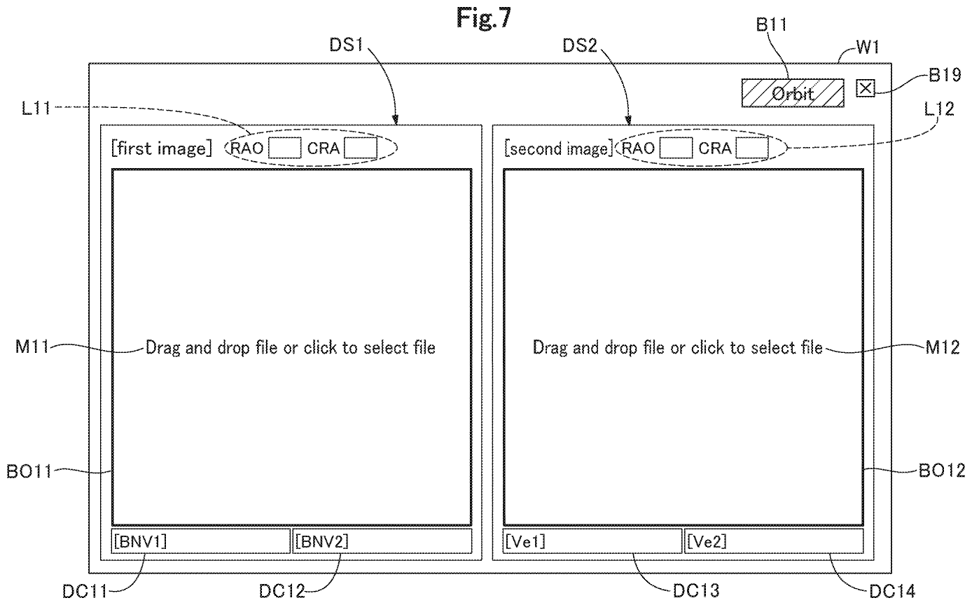

In step S10, the calculation unit 11 of the surgery assistance device 10 acquires a first image and a second image. A first screen W1 (see FIG. 7) is a screen for prompting the operator to input the first image and the second image. The “first image” is an image that has been captured with the FPD 21 in an arbitrary placement position. The “second image” is an image that has been captured with the FPD 21 in an arbitrary placement position that is different to the first position. The first image and the second image both include the target blood vessel. In the first image and the second image, the placement position of the FPD 21 is different. As a result, the target blood vessel included in the first image and the target blood vessel included in the second image show an image of the target blood vessel when viewed from different directions. The placement position of the FPD 21 when the first image is acquired is also referred to as a “first position”. The placement position of the FPD 21 when the second image is acquired is also referred to as a “second position”. In step S10 of the guide processing, the calculation unit 11 displays the first screen W1 on the display device 30 in order to acquire the first image and the second image.

FIG. 7 is a diagram showing an example of the first screen W1 in an initial state. The first screen W1 includes a first image specification portion DS1, a second image specification portion DS2, a trajectory display button B11, and a cancel button B19. In the initial state, the trajectory display button B11 is grayed out. The cancel button B19 is a button for closing the first screen W1.

The first image specification portion DS1 is a section used by the operator to specify the first image. The first image specification portion DS1 includes a first image acquisition FPD position L11, a message M11, a first input field BO11, a display field DC11, and a display field DC12. The message M11 is placed at the center of the first input field BO11, and displays, for example, “Drag and drop file or click to select file”. The second image specification portion DS2 is a section used by the operator to specify the second image. The second image specification portion DS2 includes a second image acquisition FPD position L12, a message M12, a second input field BO12, a display field DC13, and a display field DC14. The message M12 is placed at the center of the second input field BO12, and displays, for example, “Drag and drop file or click to select file”.

The operator follows the message M11, and drags and drops an input target image from a local folder onto the first input field BO11. The operator may click the first input field BO11 instead of dragging and dropping the image. In a case where the first input field BO11 has been clicked, a file selection window is opened for specifying the input target image. The input target image is a still image or a moving image captured in advance by the FPD 21. In the case of a moving image, the operator determines the scene to be captured while referring to the moving image that is automatically played in the first input field BO11. The operator selects, as the capture scene, a scene in which the target blood vessel is clearly visible, from the moving image. Then, the operator follows the message M12, and drags and drops an input target image from a local folder onto the second input field BO12, or specifying an input target image using a selection window. The details are the same as the input to the first input field BO11.

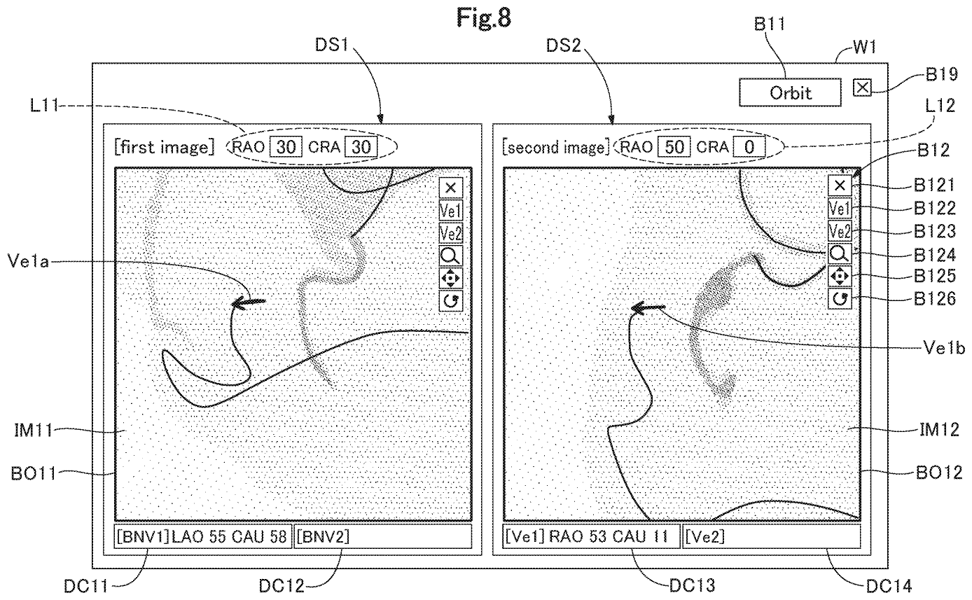

FIG. 8 is an explanatory diagram of an acquisition method of the target blood vessel using the first screen W1. The first input field BO11 shown in FIG. 8 displays the image that has been specified as the first image. Of the first image specification portion DS1, the first image acquisition FPD position L11 is the placement position of the FPD 21 when the first image was captured. In a case where the image that has been specified in the first input field BO11 includes position information of the FPD at the time of image capture, the position information of the FPD that is stored in association with the image is automatically displayed in the first image acquisition FPD position L11. In a case where the image that has been specified in the first input field BO11 does not include position information of the FPD at the time of image capture, the operator inputs the first image acquisition FPD position L11. The second input field BO12 displays the image that has been specified as the second image. Of the second image specification portion DS2, the second image acquisition FPD position L12 is the placement position of the FPD 21 when the second image was captured. The second image acquisition FPD position L12, in a similar manner to the first image, is automatically displayed, or is manually input by the operator. As shown in FIG. 8, the first image and the second image include an image of the heart 91, an image of a cardiac blood vessel, an image of the medical device that has been inserted into the cardiac blood vessel, an image of a contrast medium, and the like.

Image operation buttons B12 are displayed superimposed on the upper right of each of the first image and the second image. For convenience of the illustration, reference signs are displayed in FIG. 8 only for the image operation buttons B12 that are superimposed on the second image. The image operation buttons B12 include, in order from the top, a cancel button B121, a first vector drawing button B122, a second vector drawing button B123, a magnification button B124, a range movement button B125, and a redo button B126. The cancel button B121 is a button for discarding the selected image, and returning the first input field BO11 or the second input field BO12 to the state shown in FIG. 7. The first vector drawing button B122 is a button for the operator to draw a line segment that specifies a target blood vessel. The second vector drawing button B123 is a button for the operator to draw a line segment that specifies a target blood vessel. The target blood vessel specified by the first vector drawing button B122 and the target blood vessel specified by the second vector drawing button B123 are different blood vessels. That is, the second vector drawing button B123 is used in a case where a second target blood vessel is specified. The second vector drawing button B123 can be omitted. The magnification button B124 is a button for magnifying the image that is being displayed in the first input field BO11 or the second input field BO12. The range movement button B125 is a button for moving the center of the image that is being displayed in the first input field BO11 or the second input field BO12. The redo button B126 is a button for canceling the previous operation.

The operator, after pressing the first vector drawing button B122 of the first input field BO11, traces the target blood vessel appearing in the image displayed in the first input field BO11. Then, a first vector Ve1a representing the target blood vessel is displayed on the image in the first input field BO11. The display field DC11 displays the position of the FPD 21 when the first image acquisition FPD position L11 is rotated by 90° around the first vector Ve1a . Then, the operator, after pressing the first vector drawing button B122 of the second input field BO12, traces the target blood vessel appearing in the image displayed in the second input field BO12. For example, the operator can trace the target blood vessel using a mouse cursor. Then, a first vector Ve1b representing the target blood vessel is displayed on the image in the second input field BO12. The display field DC13 displays “a first vector Ve1 representing the vector of the blood vessel axis of the target blood vessel in the three-dimensional space in which the FPD 21 has been placed”. The first vector Ve1 in the display panel DC13 is calculated from the first vector Ve1a and the first vector Ve1b. The first vector Ve1 in the display panel DC13 is the calculation result of step S14 described below. For example, the example of FIG. 8 shows that the first vector Ve1b representing the target blood vessel is equivalent to an arrow from the origin (0, 0, 0) of the three-dimensional space in which the FPD 21 is placed toward the direction RAO 53, CAU 11. The blood vessel represented by the first vector Ve1a and the blood vessel represented by the first vector Ve1b are the same blood vessel. As shown in FIG. 8, after finishing the drawing of the first vectors Ve1a and Ve1b, the trajectory display button B11 is no longer grayed out.

In a case where the operator draws a second vector using the second vector drawing button B123, different arrows to the first vectors Ve1a and Ve1b are displayed in the first input field BO11 and the second input field BO12. The display field DC12 displays the position of the FPD 21 when the first image acquisition FPD position L11 is rotated by 90° around the second vector. In the display panel DC14, the second vector in the three-dimensional space in which the FPD 21 is placed is displayed in a representation that shows the placement position of the FPD 21. The drawing of the second vector is optional and will not be described further.

In step S10 of FIG. 6, the calculation unit 11 acquires the first image and the second image that have been specified on the first screen W1. At this time, the calculation unit 11 acquires both a “first position”, which is the placement position of the FPD 21 when the first image was captured, and a “second position”, which is the placement position of the FPD when the second image was captured. The first position is the value displayed in the first image acquisition FPD position L11. The second position is the value displayed in the second image acquisition FPD position L12. The calculation unit 11, in step S12, acquires the first vector Ve1a and the first vector Ve1b that have been specified on the first screen W1. At this time, the calculation unit 11 acquires both the angles of the first vector Ve1a and the angles of the first vector Ve1b. The angles of the first vector Ve1a are the values displayed in the display panel DC11. The angles of the first vector Ve1b are the values displayed in the display panel DC13.

In step S14, the calculation unit 11 calculates (derives) the blood vessel axis of the target blood vessel using the information acquired in steps S10 to S12. In other words, the calculation unit 11 calculates the first vector Ve1 representing the vector of the blood vessel axis of the target blood vessel in the three-dimensional space in which the FPD 21 has been placed. The first vector Ve1 is calculated from the first vectors Ve1a and Ve1b in FIG. 8 that have been drawn by the operator. The details of the method of calculating the vector of a blood vessel axis in a three-dimensional space in which an FPD has been placed using first and second images that have been captured at first and second positions, which are different positions from each other, is disclosed in International Application PCT/JP2021/034980. In International Application PCT/JP2021/034980, a blood vessel presence plane H2 viewed from a first position, and a blood vessel presence plane S viewed from a second position are used to define a straight line on which the H2 plane and the S plane intersect, and then a “blood vessel axis vector”, being the straight line, is calculated. In the example of the present embodiment, for example, the plane on which the first vector Ve1a shown by the first image exists corresponds to the H2 plane, and the first vector Ve1b shown by the second image exists corresponds to the S plane. In this state, the calculation unit 11 calculates the first vector Ve1, which is the vector of the blood vessel axis of the target blood vessel.

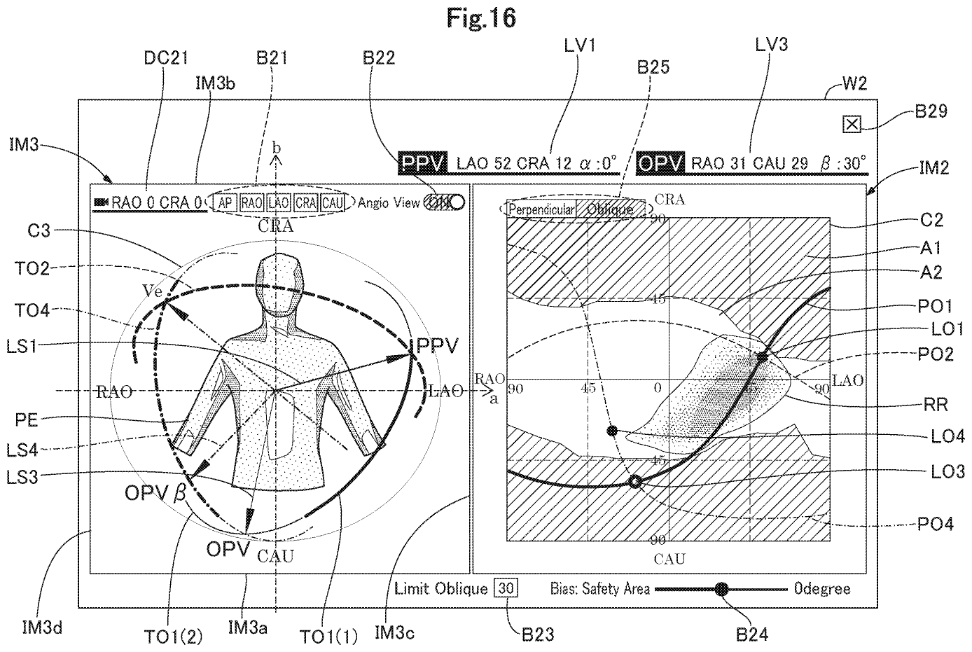

In step S16, the calculation unit 11 calculates the first planar trajectory PO1 (see FIG. 16). The first planar trajectory PO1 is a trajectory connecting points at which the placement position of the FPD 21 is positioned in a first direction, which is perpendicular to the extending direction of the blood vessel axis. The calculation unit 11 can calculate the first planar trajectory PO1 using the blood vessel axis obtained in step S14.

In step S20, the two-dimensional image generation unit 12 of the surgery assistance device 10 detects the pressing of the trajectory display button B11 on the first screen W1. After the pressing of the trajectory display button B11 is detected, the two-dimensional image generation unit 12 acquires an allowed tilt angle. The allowed tilt angle represents the allowed tilt angle of the FPD 21, and is a numerical value that indicates the upper limit value of how many degrees the FPD 21 can be tilted when placed in an arbitrary placement position. The allowed tilt angle refers to the upper limit value of α described later in (b6). That is, the allowed tilt angle is an upper limit value of the movement amount of the second position LO2 based on the first position LO1. The first position LO1 is an arbitrary placement position of the FPD 21 on the first planar trajectory PO1. The second position LO2 is an arbitrary placement position of the FPD 21 on the second planar trajectory PO2. The second planar trajectory PO2 is a trajectory connecting points at which the placement position of the FPD 21 is positioned in the second direction, which is on a first virtual plane that passes through the extending direction of the blood vessel axis Ve, and a virtual line segment that extends from the origin of the three-dimensional space in which the FPD 21 is placed, to the first position LO1. When step S20 is executed for the first time, the two-dimensional image generation unit 12 acquires an initial value of the allowed tilt angle that is set in advance in the storage unit of the surgery assistance device 10. When step S20 is executed for the second and subsequent times, the two-dimensional image generation unit 12 acquires the content that has been input to an allowed tilt angle specification field B23 of the second screen W2 (see FIG. 16) as the allowed tilt angle. The details of the second screen W2 will be described later.

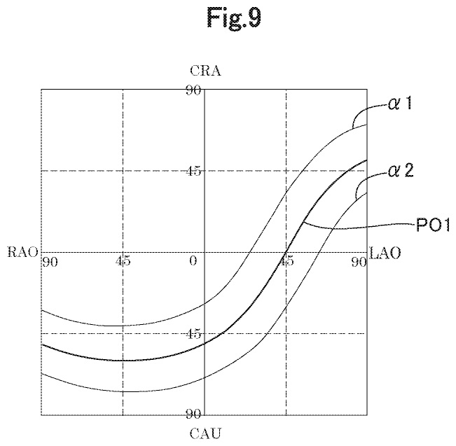

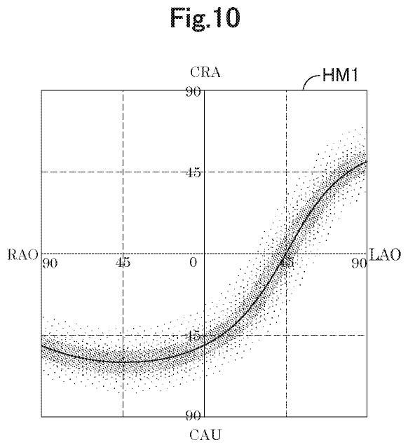

FIG. 9 is a diagram showing a generation method of a first heat map HM1. FIG. 10 is a diagram showing an example of the first heat map HM1. In step S22 of FIG. 6, the two-dimensional image generation unit 12 generates the first heat map. In FIG. 9, the first planar trajectory PO1 is drawn on a graph representing the three-dimensional space in which the FPD 21 is placed using a Mercator projection. In FIG. 9, α1 is a curve obtained by adding the allowed tilt angle acquired in step S20 to the first planar trajectory PO1. In FIG. 9, α2 is a curve obtained by subtracting the allowed tilt angle acquired in step S20 from the first planar trajectory PO1. That is, the α1 and α2 curves are straight lines that indicate the upper limit value of the allowed tilt angle.

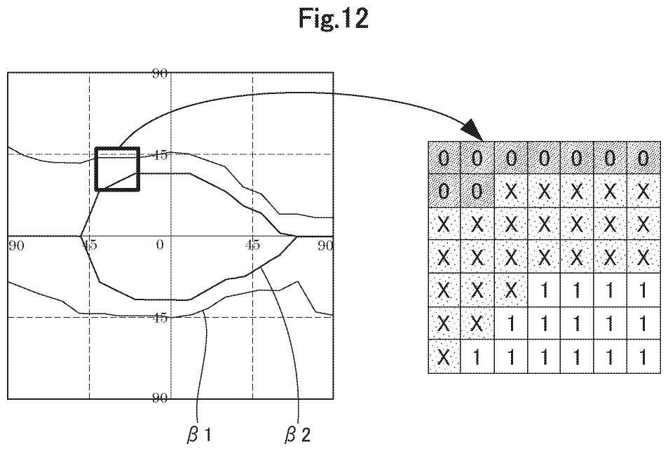

The two-dimensional image generation unit 12 divides the graph above into squares having a predetermined size, and then assigns a score to each square. Specifically, the two-dimensional image generation unit 12 assigns a score of 1 on the first planar trajectory PO1, a score that approaches 0 as the α1 curve is approached from the trajectory PO1, and a score that approaches 0 as the α2 curve is approached from the trajectory PO1. The score on the outside of the α1 curve is 0. The score on the outside of the α2 curve is 0. As a result, the two-dimensional image generation unit 12 is capable of generating the first heat map HM1 as shown in FIG. 10. In the first heat map HM1, points with relatively high scores are shown in a darker color than points with relatively low scores. That is, in the first heat map HM1, the visibility of the section far from the upper limit value of the allowed tilt angle is set higher than the visibility of the section close to the upper limit value. The section far from the upper limit value of the allowed tilt angle is, in other words, the section close to the trajectory PO1. In the first heat map HM1, the visibility changes gradually due to a gradation display. The first heat map HM1 is also referred to as a “first map”. In FIG. 9 and later diagrams, and specifically in the description of FIGS. 12 to 15, the description assumes that the graph above is divided into m×m squares. Here, m is an arbitrary natural number. The first square in the graph is also referred to as the (1, 1) position, and the last square in the graph is also referred to as the (m, m) position.

In steps S30 and S32 of FIG. 6, the two-dimensional image generation unit 12 acquires the settings of the range of motion information INa and the recommended range of motion information INb. The range of motion information INa is information indicating the range in which the FPD 21 can be moved. The range in which the FPD 21 can be moved is also referred to as a “first range”. The first range includes, in addition to a range in which the FPD 21 can be easily moved (in other words, a range in which the C-arm 23 can be easily moved), a range in which the FPD 21 can be moved with difficulty (in other words, a range in which the C-arm 23 can be moved with difficulty). The recommended range of motion information INb is information indicating the range in which the FPD 21 can be easily moved. The range in which the FPD 21 can be easily moved is also referred to as a “second range”. The range in which the FPD 21 can be easily moved (second range) is a range that is arbitrarily set by the operator inside the range in which the FPD 21 can be moved (first range). For example, the operator may set, as the second range, a range in which the FPD 21 can be smoothly moved without interfering with the progress of the procedure. Specifically, the operator may set, as the second range, a range that is set several percent inside the range in which the FPD 21 can be moved (first range). Alternatively, the operator may set, as the second range, a range corresponding to a hot point inside the range in which the FPD 21 can be moved (first range). A hot point refers to a location that is particularly recommended as the placement position of the FPD 21. The hot point will be discussed in more detail below. The entire second range is included in the first range. In step S30 of the guide processing, the two-dimensional image generation unit 12 displays a third screen W3 on the display device 30 in order to acquire the range of motion information INa and the recommended range of motion information INb.

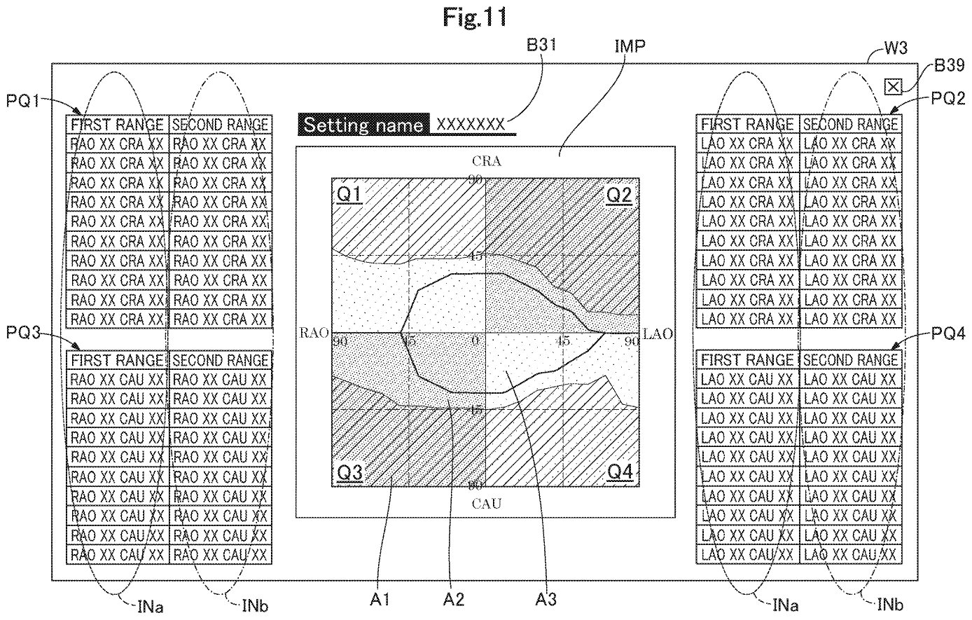

FIG. 11 is a diagram showing an example of the third screen W3. The third screen W3 includes a first range of motion specification field PQ1, a second range of motion specification field PQ2, a third range of motion specification field PQ3, a fourth range of motion specification field PQ4, a range of motion image IMP, a setting name specification field B31, and a cancel button B39.

The first range of motion specification field PQ1 is an information input field for setting the first range and the second range inside a range in an RAO-CRA direction from the origin of the three-dimensional space in which the FPD 21 has been placed. The second range of motion specification field PQ2 is an information input field for setting the first range and the second range inside a range in an LAO-CRA direction from the origin of the three-dimensional space in which the FPD 21 has been placed. The third range of motion specification field PQ3 is an information input field for setting the first range and the second range inside a range in an RAO-CAU direction from the origin of the three-dimensional space in which the FPD 21 has been placed. The fourth range of motion specification field PQ4 is an information input field for setting the first range and the second range inside a range in an LAO-CAU direction from the origin of the three-dimensional space in which the FPD 21 has been placed. In FIG. 11, the “XX” illustrated in the first to fourth range of motion specification fields PQ1 to PQ4 represent arbitrary numbers. As shown in FIG. 11, in the first to fourth range of motion specification fields PQ1 to PQ4, an outline of the first range is defined by a combination of a plurality of representative points below the label “first range”. In other words, the first range of the first to fourth range of motion specification fields PQ1 to PQ4 is input with the placement position of the FPD 21 when the FPD 21 is at a boundary position between the range in which the FPD 21 can be moved (FIG. 11: movable range A2) and the range in which the FPD 21 cannot be moved (FIG. 11: non-movable range A1). Similarly, in the first to fourth range of motion specification fields PQ1 to PQ4, an outline of the second range is defined by a combination of a plurality of representative points below the label “second range”. The second range of the first to fourth range of motion specification fields PQ1 to PQ4 is input with the placement position of the FPD 21 when the FPD 21 is at a boundary position between the range in which the FPD 21 can be easily moved (FIG. 11: easily movable range A3) and the range in which the FPD 21 can be moved (FIG. 11: movable range A2).

The range of motion image IMP is a graph that uses a Mercator projection to represent the three-dimensional space in which the FPD 21 is placed, and depicts the non-movable range A1, the movable range A2, and the easily movable range A3 defined by the first to fourth range of motion specification fields PQ1 to PQ4. The movable range A2 is the “first range” in which the FPD 21 can be moved. The easily movable range A3 is the “second range” in which the FPD 21 can be easily moved. The non-movable range A1 is the entire range outside the first range. The setting name specification field B31 is a file name specification field for naming and saving the setting contents of the current third screen W3. By naming and saving the setting contents, it becomes possible to easily read the setting contents when performing the guide processing for the second and subsequent times. For example, if the setting contents are named and saved in advance for each operator, it becomes possible to set the range of motion information INa and recommended range of motion information INb for each operator, which allows the usability of the surgery assistance device 10 to be improved. In this case, for example, the surgery assistance device 10 stores the range of motion information Ina1 and recommended range of motion information Inb1 set by a first operator, and the range of motion information Ina2 and recommended range of motion information Inb2 set by a second operator. In step S30, the first operator may specify the range of motion information Ina1 and recommended range of motion information Inb1 that he/she has saved, and then cause the two-dimensional image generation unit 12 to read the information. In this way, in a case where the setting contents of the third screen W3 are saved for each operator, range of motion information IN that does not change for each operator may be omitted.

The cancel button B39 is a button for closing the third screen W3. In step S30, instead of displaying the third screen W3 and acquiring the input content, the two-dimensional image generation unit 12 may read the setting contents of the third screen W3 that has been saved in advance, and then proceed with the processing using the setting contents. In this case, the operator completes the setting of the third screen W3 in advance of executing the guide processing of FIG. 6.

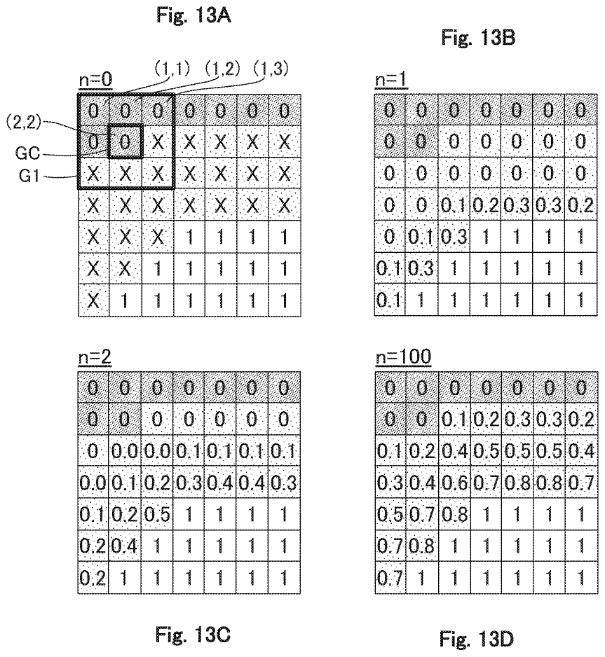

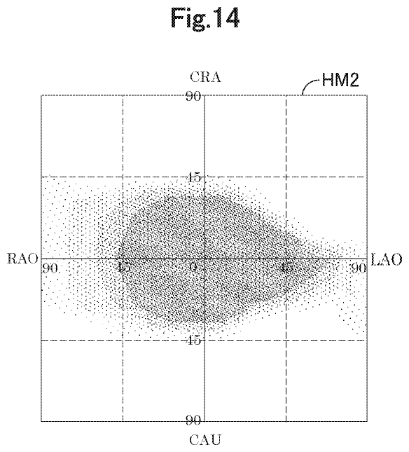

FIGS. 12 and 13 are diagrams showing a generation method of the second heat map HM2. FIG. 14 is a diagram showing an example of the second heat map HM2. In step S34 of FIG. 6, the two-dimensional image generation unit 12 generates the second heat map. In FIG. 12, a curve β1 that defines the first range and a curve β2 that defines the second range are drawn on a graph representing the three-dimensional space in which the FPD 21 is placed using a Mercator projection. In other words, the curve β1 is a boundary line between the non-movable range A1 and the movable range A2. The curve β2 is a boundary line between the movable range A2 and the easily movable range A3. As shown in the enlarged view on the right side of FIG. 12, the two-dimensional image generation unit 12 divides the graph above into squares having a predetermined size, and then assigns a score to each square. Specifically, first, the two-dimensional image generation unit 12 assigns a score of 1 to squares located in the easily movable range A3, a score of X to squares located in the movable range A2, and a score of 0 to squares located in the non-movable range A1. “X” in FIGS. 12 and 13 is a symbol that indicates an indeterminate score.

Next, as shown in FIG. 13, the two-dimensional image generation unit 12 sets the (2, 2) position of the grid of the graph as the center GC, and defines a range G1 of eight surrounding squares around the center GC. The two-dimensional image generation unit 12 sets the score of the center GC as (score of center GC 30 scores of each of the eight surrounding squares)/9. The Xs, which indicate an indeterminate score, are calculated as 0. In the illustrated example, the score of the center GC becomes (0+0+0+0+0+0+0+0+0)/9=0. The two-dimensional image generation unit 12 moves the center GC one at a time, and repeats the calculation until the end of the graph. In FIG. 13, n=1 represents the graph obtained after one cycle of the calculation described above. As a result of the calculation, the Xs, which indicate an indeterminate score, are eliminated from the graph, and numerical values are input to all of the squares. In the example of FIG. 13, for convenience of the illustration, the scores in the squares are rounded to one decimal place. The two-dimensional image generation unit 12 returns the center GC to the (2, 2) position, and repeats the same calculation n times, where n is a natural number that can be arbitrarily determined. As shown in FIG. 13, when n=100, as a result of repeating the calculation described above n times, the scores of the squares located in the movable range A2 change more gradually than when n=1. As a result, the two-dimensional image generation unit 12 is capable of generating the second heat map HM2 as shown in FIG. 14. In the second heat map HM2, points with relatively high scores are shown in a darker color than points with relatively low scores. That is, in the second heat map HM2, the visibility of the section corresponding to the easily movable range A3 is set higher than the visibility of the section corresponding to only the movable range A2. In other words, in the second heat map HM2, the visibility of the section corresponding to the second range is set higher than the visibility of the section corresponding to only the first range. In the second heat map HM2, the visibility changes gradually due to a gradation display. The second heat map HM2 is also referred to as a “second map”.

The score calculation method described in FIG. 13 is merely an example, and various modifications are possible. For example, the range G1 is set to 3×3=9 squares. The range G1 is set to m×m. Here, m is an arbitrary natural number greater than or equal to 2. For example, the number of times the repeated calculations n described above are performed can be arbitrarily set. The repeated calculations described above may be omitted. The two-dimensional image generation unit 12 can use the score calculation method (and modifications thereof) described in FIG. 13 in the generation of the first heat map HM1 described in FIGS. 9 and 10.

In step S40 of FIG. 6, the two-dimensional image generation unit 12 acquires a weight. When step S40 is executed for the first time, the two-dimensional image generation unit 12 acquires an initial value of the weight that is set in advance in the storage unit of the surgery assistance device 10. When step S40 is executed for the second and subsequent times, the two-dimensional image generation unit 12 acquires the content that has set in the weight specification field B24 of the second screen W2 as the weight. The details of the second screen W2 will be described later.

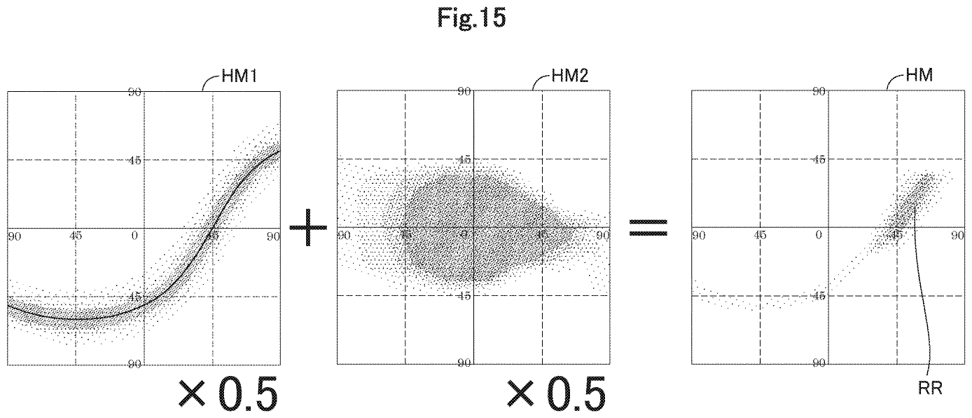

FIG. 15 is a diagram describing the summation of the first heat map HM1 and the second heat map HM2. In step S42 in FIG. 6, the two-dimensional image generation unit 12 performs the summation of the first heat map HM1 and the second heat map HM2 to generate a single composite heat map HM. Specifically, the two-dimensional image generation unit 12 firstly calculates a first value and a second value. The first value is the score of the (1, 1) position of the first heat map HM1×(1−weight). The second value is the score of the (1, 1) position of the second heat map HM2×(weight). The two-dimensional image generation unit 12 sets the score of the (1, 1) position of the composite heat map HM to the sum of the first value and the second value if both the first value and the second value are greater than zero. The two-dimensional image generation unit 12 sets the score of the (1, 1) position of the composite heat map HM to 0 if at least one of the first value and the second value is 0. For example, if the weight is 0.5, the score at the (1, 1) position of the first heat map HM1 is 0.5, and the score at the (1, 1) position of the second heat map HM2 is 0, then the score at the (1, 1) position of the composite heat map HM is 0 because the second value is 0. For example, if the weight is 0.5, the score at the (1, 1) position of the first heat map HM1 is 1, and the score at the (1, 1) position of the second heat map HM2 is 0.6, then the score at the (1, 1) position of the composite heat map HM is 0.5+0.3=0.8.

The two-dimensional image generation unit 12 moves the target position of the score calculation one position at a time from the (1, 1) position for the first heat map HM1 and the second heat map HM2, and repeats the calculation up to the (m, m) position, which is the end of the graph. The composite heat map HM obtained in this manner is depicted on the right side of FIG. 15. In the composite heat map HM, points with relatively high scores are shown in a darker color than points with relatively low scores. In the composite heat map HM, the range with a score of 0 or more is also called a “recommended range RR”. The recommended range RR indicates a range recommended as the placement position of the FPD 21. That is, the recommended range RR is calculated by weighting and combining the first heat map HM1, which increases or decreases the visibility depending on the allowed tilt angle, and the second heat map HM2, which increases or decreases the visibility based on the range of motion information INa and the recommended range of motion information INb.

In step S44 of FIG. 6, the two-dimensional image generation unit 12 calculates (derives) a recommended position. The recommended position is different from the recommended range RR, which indicates a range, and indicates a point that is recommended as the placement position of the FPD 21. The calculation method of the recommended position is described later. In step S46, the two-dimensional image generation unit 12 generates a two-dimensional image. The “two-dimensional image” is an image showing the placement position of the FPD 21 in two-dimensional coordinates. In step S46, the three-dimensional image generation unit 13 of the surgery assistance device 10 generates a three-dimensional image. The “three-dimensional image” is an image showing the placement position of the FPD 21 in three-dimensional coordinates. In step S48, the composite image generation unit 14 of the surgery assistance device 10 generates a composite image in which the two-dimensional image and the three-dimensional image generated in step S46 have been combined. The “combining” performed by the composite image generation unit 14 refers to forming a single image by arranging the two-dimensional image and the three-dimensional image side-by-side on the left and right. In step S48, the composite image generation unit 14 outputs the generated composite image to the display device 30. In other words, the composite image generation unit 14 displays the composite image on the display device 30.

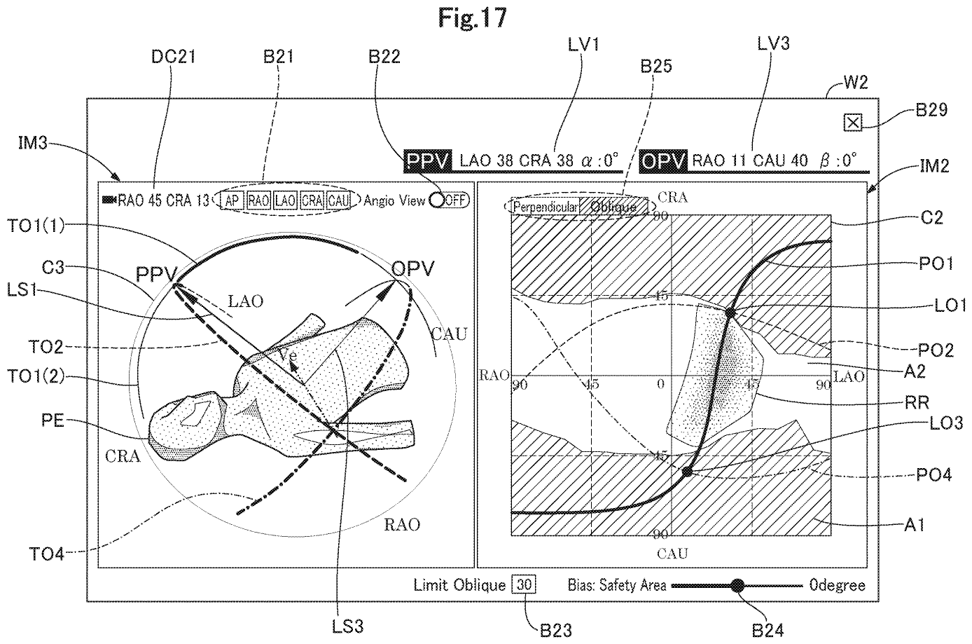

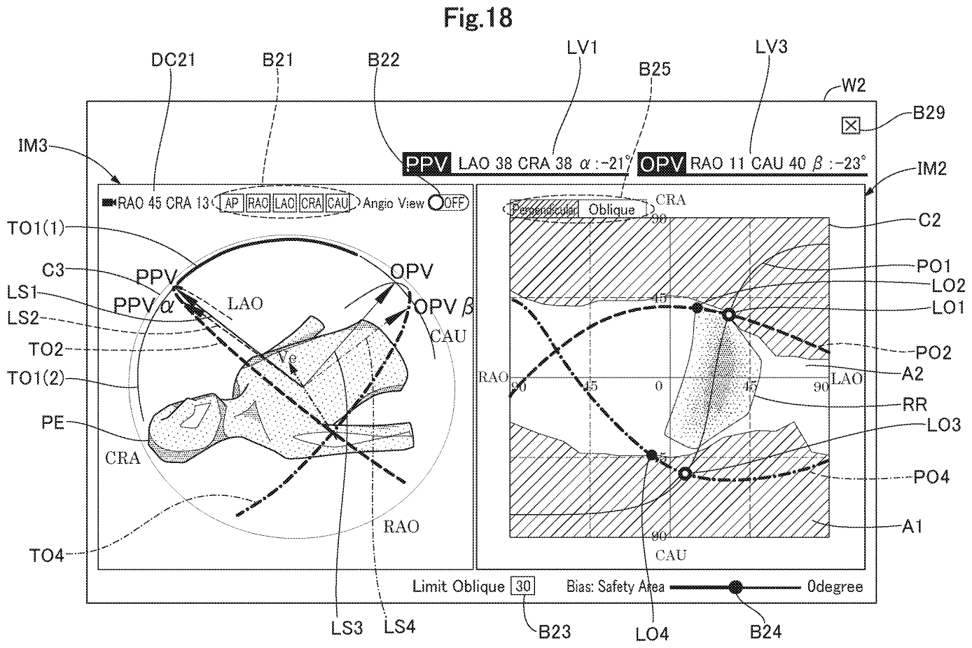

FIG. 16 is a diagram illustrating an example of a composite image. FIGS. 17 and 18 are a diagrams illustrating other examples of a composite image. Hereinafter, the screen for displaying the composite image is also referred to as a “second screen W2”. The second screen W2 includes a two-dimensional image IM2, a three-dimensional image IM3, a vertical position LV1, a normal position LV3, and a cancel button B29.

The two-dimensional image IM2 is the two-dimensional image generated by the two-dimensional image generation unit 12. The two-dimensional image IM2 includes b1 to b10 below.
(b1) Two-dimensional coordinates C2: Coordinates representing the three-dimensional space in which the FPD 21 is placed using a Mercator projection. “0” represents the origin of the two-dimensional space. The origin of the two-dimensional coordinates C2 is RAO=0, LAO=0, CRA=0, CAU=0. The two-dimensional coordinates C2 include a square grid and numbers representing the magnitude of the angles θ1 and θ2 from the origin of the two-dimensional coordinates C2 toward each of the directions LAO, RAO, CRA, and CAU described in FIGS. 2 to 5.
(b2) Non-movable range A1, movable range A2: The non-movable range A1 is a range in which the FPD 21 cannot be moved. A hatching is applied to the non-movable range A1. The non-movable range A1 may be filled in with a dark color. The movable range A2 is a range in which the FPD 21 can be moved. The movable range A2 corresponds to the first range described in FIG. 11. The movable range A2 is filled in with a white color. The movable range A2 may be filled in with a lighter color than the non-movable range A1. Also, b2 may further include the easily movable range A3 described in FIG. 11, that is, the second range. The movable range of the FPD 21 is the content set in steps S30 and S32 of FIG. 6.
(b3) First planar trajectory PO1: The first planar trajectory PO1 is a trajectory connecting points at which the placement position of the FPD 21 is positioned in a first direction, which is perpendicular to the extending direction of the blood vessel axis Ve. The blood vessel axis Ve is the first vector Ve1 obtained in step S14, which represents the vector of the blood vessel axis of the target blood vessel. The first planar trajectory PO1 is the trajectory calculated in step S16 of FIG. 6. The first planar trajectory PO1 can also be referred to as a first planar range PO1. In the illustrated example, the first planar trajectory PO1 is represented by a bold and solid curved line.
(b4) First position LO1: The first position LO1 is an arbitrary placement position of the FPD 21 on the first planar trajectory PO1. The first position LO1 can be said to be an arbitrary point on the first planar trajectory PO1. In the example of FIGS. 16 and 17, the first position LO1 is represented by a black circle. In the example of FIG. 18, the first position LO1 is represented by a hollow circle. In a case where the first position LO1 is a hollow circle, this indicates that a second position LO2 based on the first position LO1 exists (see FIG. 18).
(b5) Second planar trajectory PO2: The second planar trajectory PO2 is a trajectory connecting points at which the placement position of the FPD 21 is positioned in the second direction, which is on a first virtual plane that passes through the extending direction of the blood vessel axis Ve, and a virtual line segment that extends from the origin of the three-dimensional space in which the FPD 21 is placed, to the first position LO1. The second planar trajectory PO2 can also be referred to as a second planar range PO2. In the illustrated example, the second planar trajectory PO2 is represented by a dashed line.
(b6) Second position LO2: The second position LO2 is an arbitrary placement position of the FPD 21 on the second planar trajectory PO2. The second position LO2 can be said to be an arbitrary point on the second planar trajectory PO2. The second position LO2 represents the placement position of the FPD 21 based on the first position LO1 when the C-arm 23 is tilted in a state where the FPD 21 is positioned on the first virtual plane. The movement amount of the second position LO2 based on the first position LO1 is represented by “α”. In the example of FIGS. 16 and 17, because α=0° at the vertical position LV1, a point representing the second position LO2 is not drawn. In the example of FIG. 18, the second position LO2 is represented by a black circle.
(b7) Third position LO3: The third position LO3 is a position that is separated from the first position LO1 on the first planar trajectory PO1. The third position LO3 can be specified as follows. First, the first virtual plane is defined that passes through the extending direction of the blood vessel axis Ve, and a virtual line segment that extends from the origin of the three-dimensional space in which the FPD 21 is placed, to the first position LO1. The third position LO3 is a point on a second virtual plane, which is obtained by rotating the first virtual plane by 90° around the extending direction of the blood vessel axis Ve. The third position LO3 can be said to be a placement position of the FPD 21 that is on the first planar trajectory PO1, and separated by 90° with respect to the first position LO1. In the example of FIGS. 16 and 18, the third position LO3 is represented by a hollow circle. In the example of FIG. 17, the third position LO3 is represented by a black circle. In a case where the third position LO3 is a hollow circle, this indicates that a fourth position LO4 based on the third position LO3 exists. As is clear from FIGS. 16 and 17, the third position LO3 changes in response to a change in the position of the first position LO1. In other words, the third position LO3 moves so as to follow the first position LO1.
(b8) Fourth planar trajectory PO4: The fourth planar trajectory PO4 is a trajectory connecting points at which the placement position of the FPD 21 is positioned in a fourth direction on the second virtual plane. The second virtual plane is the same as the second virtual plane described in relation to the third position LO3. The fourth planar trajectory PO4 can also be referred to as a fourth planar range PO4. In the illustrated example, the fourth planar trajectory PO4 is represented by a dash-dotted line.
(b9) Fourth position LO4: The fourth position LO4 is an arbitrary placement position of the FPD 21 on the fourth planar trajectory PO4. The fourth position LO4 can be said to be an arbitrary point on the fourth planar trajectory PO4. The fourth position LO4 represents the placement position of the FPD 21 based on the third position LO3 when the C-arm 23 is tilted in a state where the FPD 21 is positioned on the second virtual plane. The movement amount of the fourth position LO4 based on the third position LO3 is represented by “β”. In the example of FIG. 17, because β=0° in the normal position LV3, a point representing the fourth position LO4 is not drawn. In the example of FIGS. 16 and 18, the fourth position LO4 is represented by a black circle.
(b10) Recommended range RR: The recommended range RR indicates a range recommended as the placement position of the FPD 21. The recommended range RR is displayed superimposed on the two-dimensional coordinates C2. As described in FIG. 15, in the recommended range RR, points with relatively high scores are represented by a darker color than points with relatively low scores. That is, in the recommended range RR, points with a high recommendation level as the placement position of the FPD 21 are expressed in a relatively dark color, making the points relatively stand out. Here, “stand out” means that the visibility is high. The recommended range RR is the range calculated in step S42 of FIG. 6. As described above, the recommended range RR is calculated by weighting and combining the first heat map HM1 and the second heat map HM2. In the first heat map HM1, the visibility of the section far from the upper limit value of the allowed tilt angle is set higher than the visibility of the section close to the upper limit value. In the second heat map HM2, the visibility of the section corresponding to the second range is set higher than the visibility of the section corresponding to only the first range. As a result, in the recommended range RR, in the same manner as in the first heat map HM1, the visibility of the section far from the upper limit value of the allowed tilt angle is higher than the visibility of the section close to the upper limit value. Further, in the recommended range RR, in the same manner as in the second heat map HM2, the visibility of the section corresponding to the second range is higher than the visibility of the section corresponding to only the first range.

The other elements included in the two-dimensional image IM2 will be described. The mode specification button B25 is a button for switching between a Perpendicular mode and an Oblique mode. In the case of the Perpendicular mode, the first planar trajectory PO1 is shown with bold lines, and the operator is capable of moving the first position LO1 and the third position LO3 using the mouse. For example, in a case where the first position LO1 is moved, the operator moves the first position LO1 on the first planar trajectory PO1 by moving the mouse cursor in an arbitrary direction of the first planar trajectory PO1 in a state where the point representing the original first position LO1 is selected. In the case of the oblique mode, the second planar trajectory PO2 and the fourth planar trajectory PO4 are shown with bold lines, and the operator is capable of moving the second position LO2 and the fourth position LO4 using the mouse. In the Oblique mode, the first position LO1 and the third position LO3 are fixed.

The three-dimensional image IM3 is a three-dimensional image generated by the three-dimensional image generation unit 13. The three-dimensional image IM3 includes c1 to c9 below.
(c1) Three-dimensional coordinates C3: Coordinates representing the three-dimensional space in which the FPD 21 is placed, and includes a virtual spherical model having a predetermined radius. The spherical model has the origin (center) at the center of rotation of the C-arm 23, and is a spherical model having a radius such that the FPD 21 fixed to the C-arm 23 moves along the surface of the sphere. The spherical model may, for example, be divided into a grid having a predetermined size such as on a globe.

(c2) Patient image PE, blood vessel axis Ve: A three-dimensional model image of the human body 90. The head portion of the human body 90 is positioned in the CRA direction. The blood vessel axis Ve is displayed superimposed on the patient image PE. The blood vessel axis Ve is an arrow passing through the origin of the three-dimensional coordinates C3, and extending in the direction calculated in step S14 of FIG. 6. That is, the origin of the three-dimensional coordinates C3 is the center of rotation of the C-arm 23 (in other words, the origin of the three-dimensional space in which the FPD 21 is placed), and can be said to be an arbitrary point on the target blood vessel. In the illustrated example, the blood vessel axis Ve is represented by a two-dot chain arrow. The patient image PE is also referred to as a “patient still image”.

(c3) First three-dimensional trajectory TO1: The first three-dimensional trajectory TO1 is a trajectory representing the first planar trajectory PO1 on the spherical model in the three-dimensional coordinates C3. The first three-dimensional trajectory TO1 can also be referred to as a first three-dimensional range TO1. The first three-dimensional trajectory TO1 represents the first planar trajectory PO1 as a circumference of a circle having a radius of a predetermined length from the origin of the three-dimensional coordinates C3. As illustrated by TO1(1) in FIGS. 16 to 18, the section of the first three-dimensional trajectory TO1 that overlaps the movable range A2 of the FPD 21 is illustrated with a bold solid line. As illustrated by TO1(2) in FIGS. 16 to 18, the section of the first three-dimensional trajectory TO1 that overlaps the non-movable range A1 of the FPD 21 is represented by a thin solid line.
(c4) First line segment LS1: The first line segment LS1 is an arrow showing the first position LO1 on the first planar trajectory PO1 as a line segment extending from the origin of the three-dimensional coordinates C3 to the first position LO1. That is, the first line segment LS1 and the first position LO1 correspond to each other. In the illustrated example, the first line segment LS1 is represented by a bold solid arrow. The front end side of the arrow of the first line segment LS1 is shown with a character string “PPV”.
(c5) Second three-dimensional trajectory TO2: The second three-dimensional trajectory TO2 is a trajectory representing the second planar trajectory PO2 on the spherical model in the three-dimensional coordinates C3. The second three-dimensional trajectory TO2 can also be referred to as a second three-dimensional range TO2. The second three-dimensional trajectory TO2 represents the second planar trajectory PO2 as a circumference of a circle including the first virtual plane, and having a radius of a predetermined length from the origin of the three-dimensional coordinates C3. On the second three-dimensional trajectory TO2, the section overlapping the movable range A2 of the FPD 21 is illustrated with a bold dashed line, and the section overlapping the non-movable range A1 of the FPD 21 is represented by a thin dashed line.
(c6) Second line segment LS2: The second line segment LS2 is an arrow showing the second position LO2 on the second planar trajectory PO2 as a line segment extending from the origin of the three-dimensional coordinates C3 to the second position LO2. That is, the second line segment LS2 and the second position LO2 correspond to each other. In the example of FIGS. 16 and 17, because α=0° , the second line segment LS2 is not drawn. In the example of FIG. 18, the second line segment LS2 is represented by a dashed arrow. The front end side of the arrow of the second line segment LS2 is shown with a character string “PPVα”.
(c7) Third line segment LS3: The third line segment LS3 is an arrow showing the third position LO3 on the first planar trajectory PO1 as a line segment extending from the origin of the three-dimensional coordinates C3 to the third position LO3. That is, the third line segment LS3 and the third position LO3 correspond to each other. In the illustrated example, the third line segment LS3 is represented by a thin solid arrow. The front end side of the arrow of the third line segment LS3 is shown with a character string “OPV”.
(c8) Fourth three-dimensional trajectory TO4: The fourth three-dimensional trajectory TO4 is a trajectory representing the fourth planar trajectory PO4 on the spherical model in the three-dimensional coordinates C3. The fourth three-dimensional trajectory TO4 can also be referred to as a fourth three-dimensional range TO4. The fourth three-dimensional trajectory TO4 represents the fourth planar trajectory PO4 as a circumference of a circle including the second virtual plane, and having a radius of a predetermined length from the origin of the three-dimensional coordinates C3. On the fourth three-dimensional trajectory TO4, the section overlapping the movable range A2 of the FPD 21 is illustrated with a bold dash-dotted line, and the section overlapping the non-movable range A1 of the FPD 21 is represented by a thin dash-dotted line.
(c9) Fourth line segment LS4: The fourth line segment LS4 is an arrow showing the fourth position LO4 on the fourth planar trajectory PO4 as a line segment extending from the origin of the three-dimensional coordinates C3 to the fourth position LO4. That is, the fourth line segment LS4 and the fourth position LO4 correspond to each other. In the example of FIG. 17, because β=0° , the fourth line segment LS4 is not drawn. In the example of FIGS. 16 and 18, the fourth line segment LS4 is represented by a dash-dotted arrow. The front end side of the arrow of the fourth line segment LS4 is shown with a character string “OPVβ”.

The other elements included in the three-dimensional image IM3 will be described. The display panel DC21 displays the viewpoint position that is being currently displayed in three-dimensional coordinates C3. For example, in the example of FIG. 16, assuming that the FPD 21 moves on the surface of a virtual sphere of radius 1 in a three-dimensional space, the viewpoint is from (x, y, z)=(0, 1, 0), and therefore, RAO 0 and CRA 0 are displayed.

The viewpoint change buttons B21 are buttons for changing the viewpoint that is displayed in the three-dimensional coordinates C3. By pressing the AP button, the viewpoint becomes a view of the three-dimensional coordinates C3 and the patient image PE from the FPD 21 placed at LAO=0, RAO=0, CRA=0, and CAU=0. By pressing the RAO button, the viewpoint becomes a view of the three-dimensional coordinates C3 and the patient image PE from the RAO direction. “From the RAO direction” may be a view where RAO=90°, or may be approximately RAO=70° to match the viewpoint from which the operator views the patient. The same also applies to the LAO, CRA, and CAU directions. By pressing the LAO button, the viewpoint becomes a view of the three-dimensional coordinates C3 and the patient image PE from the LAO direction. By pressing the CRA button, the viewpoint becomes a view of the three-dimensional coordinates C3 and the patient image PE from the CRA direction. By pressing the CAU button, the viewpoint becomes a view of the three-dimensional coordinates C3 and the patient image PE from the CAU direction. In addition to changing the viewpoint by pressing these buttons, the operator is capable of changing the viewpoint by moving the mouse cursor in a state where an arbitrary point in the three-dimensional coordinates C3 is selected, and then rotating the three-dimensional coordinates C3 and the patient image PE in an arbitrary direction. The first to fourth three-dimensional trajectories TO1 to TO4 and the first to fourth line segments LS1 to LS4 also rotate with the rotation of the three-dimensional coordinates C3 and the patient image PE.

The head portion position fixing button B22 can be switched ON/OFF. In a case where the head portion position fixing button B22 is in the ON state, the head portion of the patient image PE becomes fixed. As shown by the dashed arrow in FIG. 16, in the three-dimensional image IM3, a-b coordinates, which are two-dimensional coordinates passing through the origin of the three-dimensional coordinates C3, are virtually defined. As shown in FIG. 16, the three-dimensional image IM3 has a bottom side IM3a, a top side IM3b, a right side IM3c, and a left side IM3d, and has a rectangular shape in which all of the internal angles are 90°. A rectangular shape conceptually includes a square shape, in which the lengths of the four sides Im3a to IM3d are equal. The a-axis of the a-b coordinates is parallel to the bottom side IM3a of the three-dimensional image IM3. The positive a-axis direction is the left hand direction of the patient image PE laying down, which is displayed in the three-dimensional image IM3 when the AP button of the viewpoint change buttons B21 is pressed. In the example of FIG. 16, the positive a-axis direction is the direction of the right side IM3c of the three-dimensional image IM3. The b-axis of the a-b coordinates is perpendicular to the a-axis. The positive b-axis direction is the direction of the head portion of the patient image PE laying down, which is displayed in the three-dimensional image IM3 when the AP button of the viewpoint change buttons B21 is pressed. In the example of FIG. 16, the positive b-axis direction is the direction of the top side IM3b of the three-dimensional image IM3.

In a case where the operator performs an operation that rotates the patient image PE when the head portion position fixing button B22 is in the ON state, the patient image PE is rotated in a state where the head portion is fixed on the b axis with b ≥0. In other words, when the head portion position fixing button B22 is in the ON state, the operator is capable of rotating the patient image PE and the three-dimensional coordinates C3 on the a-b plane, and further, is capable of performing three-dimensional rotation in a direction passing through the a-b plane. In a case where the head portion position fixing button B22 is in the OFF state, as shown in FIGS. 17 and 18, the head portion of the patient image PE becomes movable in an arbitrary direction according to an operation by the operator. In the present embodiment, the “crown portion” of the head in the patient image PE is fixed, and the section below the crown portion rotates. The entire head portion in the patient image PE may be fixed, and the section below the head may be rotatable.

In the Perpendicular mode, the operator moves the first position LO1 displayed on the first planar trajectory PO1 while referring to the first planar trajectory PO1 and the movable range A2 displayed in the two-dimensional image IM2. Then, in the three-dimensional image IM3, the front end position of the arrow of the first line segment LS1 moves so as to follow the change in the first position LO1. That is, the first position LO1 and the first line segment LS1 are linked to each other. As a result, by referring to the three-dimensional image IM3, the operator can immediately grasp where the first position LO1 that has been specified in the two-dimensional image IM2 is positioned in the three-dimensional coordinates C3 corresponding to the actual three-dimensional space (the three-dimensional space in which the FPD 21 is placed).

In a case where the first position LO1 is moved, the third position LO3 moves in the two-dimensional image IM2, and the front end position of the arrow of the third line segment LS3 moves in the three-dimensional image IM3 so as to follow the movement of the first position LO1. That is, the third position LO3 and the third line segment LS3 are linked to each other. As a result, the operator may move the third position LO3 instead of moving the first position LO1. In a case where the third position LO3 is moved, the first position LO1 moves in the two-dimensional image IM2, and the front end position of the arrow of the first line segment LS1 moves in the three-dimensional image IM3 so as to follow the movement of the third position LO3.

In the Oblique mode, the operator moves the second position LO2 displayed on the second planar trajectory PO2 while referring to the second planar trajectory PO2 and the movable range A2 displayed in the two-dimensional image IM2. At this time, the first position LO1 changes from a black circle to a hollow circle. Then, in the three-dimensional image IM3, the front end position of the arrow of the second line segment LS2 moves so as to follow the change in the second position LO2. That is, the second position LO2 and the second line segment LS2 are linked to each other. As a result, by referring to the three-dimensional image IM3, the operator can immediately grasp where the second position LO2 that has been specified in the two-dimensional image IM2 is positioned in the three-dimensional coordinates C3 corresponding to the actual three-dimensional space.

In the Oblique mode, the operator moves the fourth position LO4 displayed on the fourth planar trajectory PO4 while referring to the fourth planar trajectory PO4 and the movable range A2 displayed in the two-dimensional image IM2. At this time, the third position LO3 changes from a black circle to a hollow circle. Then, in the three-dimensional image IM3, the front end position of the arrow of the fourth line segment LS4 moves so as to follow the change in the fourth position LO4. That is, the fourth position LO4 and the fourth line segment LS4 are linked to each other. As a result, by referring to the three-dimensional image IM3, the operator can immediately grasp where the fourth position LO4 that has been specified in the two-dimensional image IM2 is positioned in the three-dimensional coordinates C3 corresponding to the actual three-dimensional space.

The vertical position LV1 is a character string indicating the placement position of the FPD 21 that is specified by either one of the first position LO1 and the second position LO2. In a case where the first position LO1 is selected, the vertical position LV1 displays the angle information “LAO 52 CRA 12” of the first position LO1, and 0° as the α value. In a case where the second position LO2 is selected, the vertical position LV1 displays the angle information of the second position LO2, and a “value indicating how far away the second position LO2 is from the first position LO1” as the α value. The vertical position LV1 is also referred to as a “first character string”. The normal position LV3 is a character string indicating the placement position of the FPD 21 that is specified by either one of the third position LO3 and the fourth position LO4. In a case where the third position LO3 is selected, the normal position LV3 displays the angle information “LAO 31 CAU 29” of the third position LO3, and 0° as the β value. In a case where the fourth position LO4 is selected, the normal position LV3 displays the angle information of the fourth position LO4, and a “value indicating how far away the fourth position LO4 is from the third position LO3” as the β value. The normal position LV3 is also referred to as a “second character string”.

In the initial state of the second screen W2, the recommended position calculated in step S44 of FIG. 6 is displayed in the vertical position LV1 and the normal position LV3. In the initial state of the second screen W2, the first position LO1 and the second position LO2 are aligned with the point indicated by the vertical position LV1 of the recommended position calculated in step S44 of FIG. 6. In the initial state of the second screen W2, the third position LO3 and the fourth position LO4 are aligned with the point indicated by the normal position LV3 of the recommended position calculated in step S44 of FIG. 6. In the initial state of the second screen W2, the first line segment LS1 and the second line segment LS2 are aligned with the arrow pointing to the vertical position LV1 of the recommended position calculated in step S44 of FIG. 6. In the initial state of the second screen W2, the third line segment LS3 and the fourth line segment LS4 are aligned with the arrow pointing to the normal position LV3 of the recommended position calculated in step S44 of FIG. 6.

The two-dimensional image generation unit 12 detects a change in the first to fourth positions LO1 to LO4 in the two-dimensional image IM2. In a case where a position has changed the two-dimensional image generation unit 12 generates a two-dimensional image IM2 reflecting the position after the change. Similarly, the three-dimensional image generation unit 13 generates a three-dimensional image IM3 reflecting the position after the change. The composite image generation unit 14 generates and outputs a newly generated composite image in which the two-dimensional image IM2 and the three-dimensional image IM3 have been combined, that is, the second screen W2. In this way, when the operator performs an operation to move at least one of the first to fourth positions LO1 to LO4 in the two-dimensional image IM2, the values of the vertical position LV1 and the normal position LV3 change to values corresponding to the operation content performed by the operator.

The allowed tilt angle specification field B23 is an input field for the operator to set the allowed tilt angle. The operator is capable of setting the allowed tilt angle by changing the numerical value in a text box. In the initial state of the second screen W2, the allowed tilt angle specification field B23 displays the initial value used in step S20 of FIG. 6.

The weight specification field B24 is an input field for the operator to set the weight. The operator is capable of setting the weight by sliding a slider bar. The Safety Area side represents a relatively large weight of the second heat map HM2 generated using the range of motion information INa and the recommended range of motion information INb. The 0 degree side represents a relatively large weight of the first heat map HM1 generated using the allowed tilt angle. The specific numerical value of the weight changes according to the movement amount of the bar from the center toward the left and right. In the initial state of the second screen W2, the weight specification field B24 displays the initial value used in step S40 of FIG. 6.

The description will be continued returning to FIG. 6. In step S50, the two-dimensional image generation unit 12 determines whether or not at least one of the allowed tilt angle condition and the weight condition has changed. In a case where the allowed tilt angle condition has changed, or more specifically, in a case where the numerical value inside the allowed tilt angle specification field B23 has changed (step S50, YES: allowed tilt angle), the two-dimensional image generation unit 12 shifts the processing to step S20. In a case where the weight condition has changed, or more specifically, in a case where the position of the slider bar of the weight specification field B24 has changed (step S50, YES: weight), the two-dimensional image generation unit 12 transitions the shifts to step S40. In a case where both the allowed tilt angle condition and the weight condition have changed, the processing shifts to step S20. As a result, the two-dimensional image generation unit 12 uses the new conditions set by the allowed tilt angle specification field B23 and the weight specification field B24 to recalculate the recommended range RR, and also regenerates the two-dimensional image IM2 including the recalculated recommended range RR. In step S60, the two-dimensional image generation unit 12 determines whether or not a termination condition has been met. In a case where the termination condition has been met (step S60: YES), the two-dimensional image generation unit 12 ends the processing. The termination condition can be arbitrarily set. The termination condition, for example, can be set as a case where pressing of the cancel button B29 has been detected.

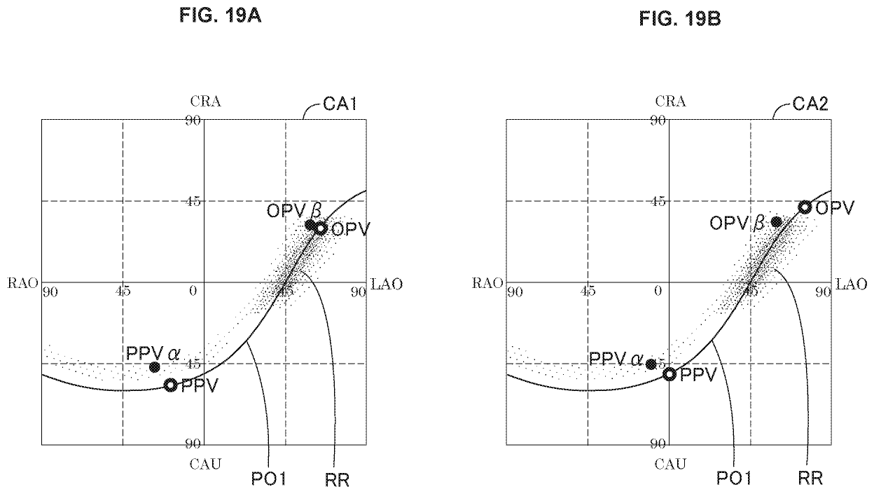

FIG. 19 is a diagram describing a calculation method of the recommended position in step S44 of FIG. 6. The two-dimensional image generation unit 12, for example, calculates the recommended position by the following procedure d1 to d3. (d1) The two-dimensional image generation unit 12 generates a plurality of candidates of the vertical position LV1 and normal position LV3. At this time, the two-dimensional image generation unit 12 includes at least one of the first position LO1 and the second position LO2 in the recommended range RR, and includes at least one of the third position LO3 and the fourth position LO4 in the recommended range RR. FIG. 19 shows a first candidate CA1 and a second candidate CA2. The first candidate CA1 and the second candidate CA2 are an example of candidates generated by the two-dimensional image generation unit 12. The first candidate CA1 and the second candidate CA2 each list, with a point representing the position, a character string “PPV” indicating that the point is the first position LO1, a character string “PPVα” indicating that the position is the second position LO2, a character string “OPV” indicating that the position is the third position LO3, and a character string “OPVβ” indicating that the position is the fourth position LO4. The first planar trajectory PO1 drawn in the first candidate CA1 and the second candidate CA2 is shown merely for convenience of the description.
(d2) The two-dimensional image generation unit 12 calculates (derives) a score with respect to each candidate generated in procedure d1. The score is calculated using a numerical value representing how close PPVα, which represents the second position LO2, and OPVβ, which represents the fourth position LO4, are to the first planar trajectory PO1. For example, in a case where PPVα is located on the first planar trajectory PO1, the PPVα score is 1. The PPVα gradually becomes smaller as the distance from the first planar trajectory PO1 increases. At the point of the allowed tilt angle, the PPVα score is 0. The two-dimensional image generation unit 12 calculates a PPVα score and an OPVβ score with respect to each candidate generated in procedure d1. The two-dimensional image generation unit 12 uses the sum of the PPVα score and OPVβ score as the score of the candidate.
(d3) The two-dimensional image generation unit 12 selects one candidate from the plurality of candidates that has the highest score calculated in procedure d2. The two-dimensional image generation unit 12 uses the combination of the vertical position LV1 and the normal position LV3 represented by the selected candidate as the recommended position. In this way, the two-dimensional image generation unit 12 includes at least one of the first position LO1 and the second position LO2 in the recommended range RR, includes at least one of the third position LO3 and the fourth position LO4 in the recommended range RR, and further, is capable of using the tilt angle of the FPD 21 represented by the second position LO2 and the tilt angle of the FPD 21 represented by the fourth position LO4 that give the smallest sum as the recommended position.

As described above, according to the surgery assistance device 10, the two-dimensional image generation unit 12, which serves as the image generation unit, generates the two-dimensional image IM2 that includes the recommended range RR that indicates the range recommended as the placement position of the FPD 21 in addition to the first planar trajectory PO1. The recommended range RR is calculated, as shown in steps S30 to S34 of FIG. 6, using the range of motion information INa that defines the first range, in which the FPD 21 can be moved, and the first planar trajectory PO1. As a result, the operator is capable of accurately and efficiently deriving the placement position of the FPD 21 by referring to the recommended range RR.

Further, according to the surgery assistance device 10, the two-dimensional image generation unit 12 sets, in the recommended range RR, the visibility of the section corresponding to the second range to be higher than the visibility of the section corresponding to only the first range. In the section corresponding to the second range, the operator is capable of easily moving the FPD 21. In the section corresponding to only the first range, although not easy, the operator is capable of moving the FPD 21. As a result, the operator is capable of accurately and efficiently deriving an appropriate and reasonable placement position of the FPD 21 by referring to the recommended range RR.

In addition, according to the surgery assistance device 10, as described in FIG. 11, the two-dimensional image generation unit 12 can also calculate (derive) the recommended range using the one recommended range of motion information INb among the plurality of recommended range of motion information INb that has been specified by the operator. Consequently, it is possible to use the recommended range of motion information INb which sets the second range that is preferred by each operator. As a result, the usability of the surgery assistance device 10 can be further improved.

Further, according to the surgery assistance device 10, the two-dimensional image generation unit 12 sets, in the recommended range RR, the visibility of the section far from the upper limit value of the allowed tilt angle to be higher than the visibility of the section close to the upper limit value. Specifically, as described in steps S20 to S42 of FIG. 6, the two-dimensional image generation unit 12 calculates (derives) the recommended range RR by weighting and combining the first heat map HM1, which increases or decreases the visibility based on the allowed tilt angle, and the second heat map HM2, which increases or decreases the visibility based on the recommended range of motion information INb. As a result, the operator is capable of accurately and efficiently deriving an optimal placement position of the FPD 21 by referring to the recommended range RR.

Also, according to the surgery assistance device 10, the two-dimensional image generation unit 12 calculates (derives) the recommended range RR by acquiring an angle set by the operator as the allowed tilt angle. As a result, the usability of the surgery assistance device 10 can be further improved.

In addition, according to the surgery assistance device 10, the two-dimensional image generation unit 12 calculates (derives) the recommended range RR by combining the first heat map HM1 and the second heat map HM2 using a weight set by the operator. As a result, the usability of the surgery assistance device 10 can be further improved.

Further, according to the surgery assistance device 10, the two-dimensional image generation unit 12 generates the two-dimensional image IM2 that includes the vertical position LV1, which represents a position of the FPD 21 for placing the FPD 21 at either one of the first position LO1 and the second position LO2, and the normal position LV3, which represents a position of the FPD 21 for placing the FPD 21 at either one of the third position LO3 and the fourth position LO4. As a result, it becomes easier for the operator to use the vertical position LV1 and the normal position LV3 and specify the position of the FPD 21 with respect to the radiation imaging device 20.

Also, according to the surgery assistance device 10, the two-dimensional image generation unit 12 sets, as the vertical position LV1 and the normal position LV3 displayed as the initial state, the tilt angle of the FPD 21 represented by the second position LO2, and the tilt angle of the FPD 21 represented by the fourth position LO4 that give the smallest sum, which is the one candidate selected from among the plurality of candidates CA1 and CA2. For this reason, the two-dimensional image generation unit 12 is capable of proposing the recommended position to the operator. The recommended position is the vertical position LV1 and the normal position LV3 displayed as the initial state.

In addition, according to the surgery assistance device 10, the three-dimensional image generation unit 13 generates the three-dimensional image IM3 that represents the placement position of the FPD 21 in the three-dimensional coordinates C3, the three-dimensional image IM3 including the blood vessel axis Ve and the first three-dimensional trajectory TO1. The composite image generation unit 14 generates and outputs the second screen W2 serving as a composite image, in which the two-dimensional image IM2 and the three-dimensional image IM3 have been combined. As a result, the operator can directly grasp the trajectory of the placement position of the FPD 21 in the actual three-dimensional space by referring to not only the two-dimensional image IM2, but also the three-dimensional image IM3. As a result, the operator is capable of accurately and efficiently deriving the optimal placement position of the FPD 21.

### Second Embodiment

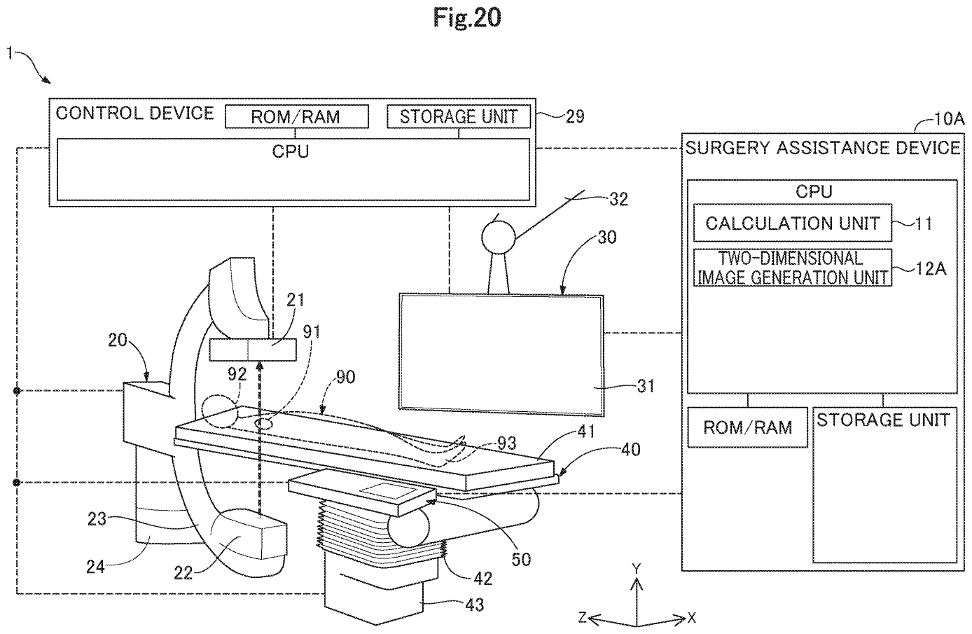

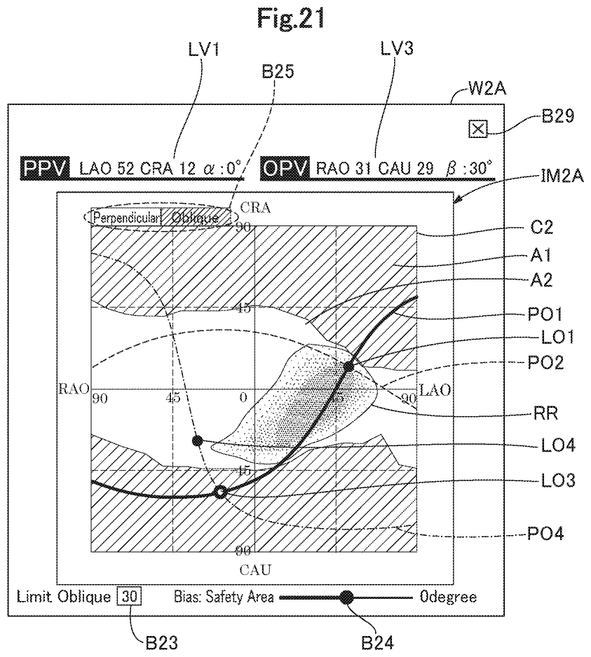

FIG. 20 is an explanatory diagram showing an example of a surgery assistance system 1 according to a second embodiment. The surgery assistance system 1 according to the second embodiment includes a surgery assistance device 10A instead of the surgery assistance device 10 described in the first embodiment. The surgery assistance device 10A includes a two-dimensional image generation unit 12A instead of the two-dimensional image generation unit 12. The two-dimensional image generation unit 12A generates the two-dimensional image IM2A shown in FIG. 21. The surgery assistance device 10A does not include the three-dimensional image generation unit 13 and the composite image generation unit 14 described in the first embodiment.
FIG. 21 is a diagram showing an example of the two-dimensional image IM2A according to the second embodiment. The second screen W2A of the present embodiment includes a two-dimensional image IM2A, the vertical position LV1, the normal position LV3, the allowed tilt angle specification field B23, and the weight specification field B24. In other words, the second screen W2A does not include the three-dimensional image IM3 described in the first embodiment.

In this way, various modifications are possible to the form of the second screen W2A. The second screen W2A may be of a form that does not include the three-dimensional image IM3, and includes only the two-dimensional image IM2A and information associated with the two-dimensional image IM2A. The surgery assistance device 10A according to the second embodiment is capable of achieving the same effects as those of the first embodiment described above.

### Third Embodiment

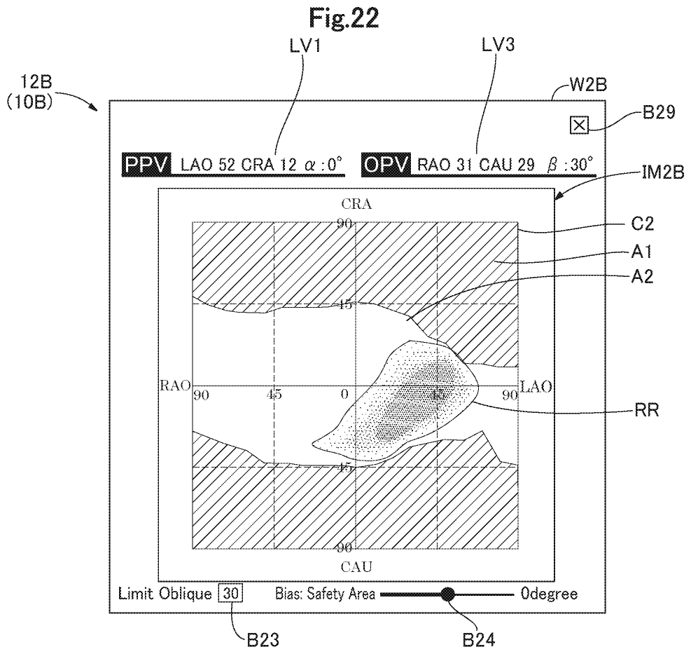

FIG. 22 is a diagram showing an example of the two-dimensional image IM2B according to a third embodiment. The surgery assistance system 1 according to the third embodiment includes a surgery assistance device 10B instead of the surgery assistance device 10A described in the second embodiment. The surgery assistance device 10B includes a two-dimensional image generation unit 12B instead of the two-dimensional image generation unit 12A. The two-dimensional image generation unit 12B generates the two-dimensional image IM2B shown in FIG. 22. The surgery assistance device 10B does not include the three-dimensional image generation unit 13 and the composite image generation unit 14 described in the first embodiment.

The second screen W2B of the present embodiment includes a two-dimensional image IM2B, the vertical position LV1, the normal position LV3, the allowed tilt angle specification field B23, and the weight specification field B24. In other words, the second screen W2B does not include the three-dimensional image IM3 described in the first embodiment. The two-dimensional image IM2B further includes the two-dimensional coordinates C2 described in the first embodiment, and only the non-movable range A1, the movable range A2, and the recommended range RR. In other words, the two-dimensional image IM2B does not include the elements b3 to b9 described in the first embodiment.

In this way, various modifications are possible to the form of the two-dimensional image IM2B. The two-dimensional image IM2B may be of a form in which at least one of the elements b2, and b3 to b9 described in the first embodiment has been omitted. For example, the two-dimensional image IM2B may be of a form in which information relating to OPV, that is, the third position LO3, the fourth planar trajectory PO4, and the fourth position LO4 have been omitted. In this case, the normal position LV3 of the second screen W2B may also be omitted.

### Fourth Embodiment

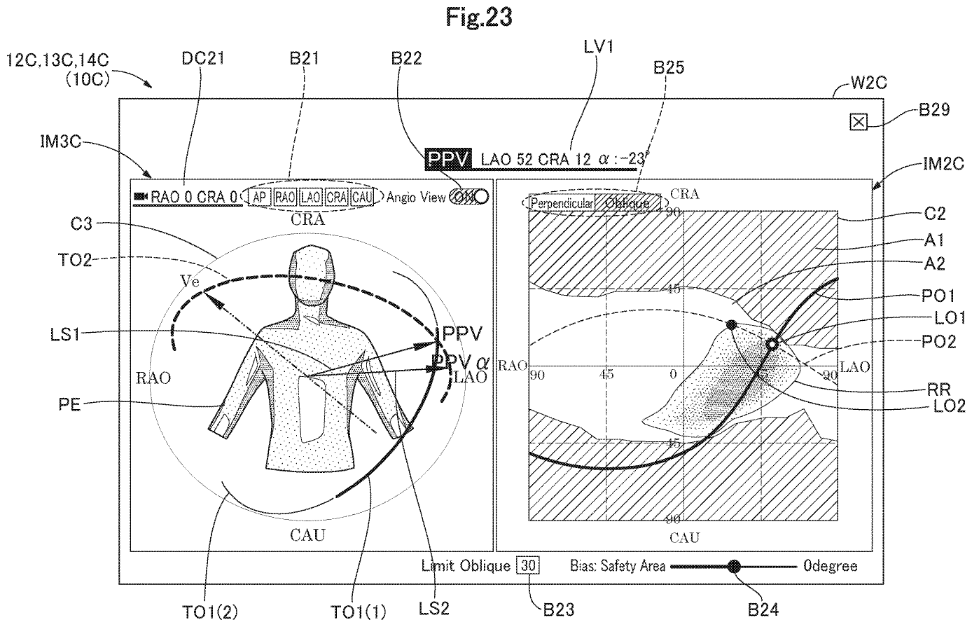

FIG. 23 is a diagram showing an example of a composite image according to a fourth embodiment. The surgery assistance system 1 according to the fourth embodiment includes a surgery assistance device 10C instead of the surgery assistance device 10 described in the first embodiment. The surgery assistance device 10C includes a two-dimensional image generation unit 12C instead of the two-dimensional image generation unit 12, a three-dimensional image generation unit 13C instead of the three-dimensional image generation unit 13, and a composite image generation unit 14C instead of the composite image generation unit 14. The two-dimensional image generation unit 12C generates the two-dimensional image IM2C shown in FIG. 23. The three-dimensional image generation unit 13C generates the three-dimensional image IM3C shown in FIG. 23. The composite image generation unit 14C generates and outputs the second screen W2C shown in FIG. 23.

The two-dimensional image IM2C does not include the third position LO3, the fourth planar trajectory PO4, and the fourth position LO4 described in the first embodiment. The three-dimensional image IM3C does not include the third line segment LS3, the fourth three-dimensional trajectory TO4, and the fourth line segment LS4 described in the first embodiment. The second screen W2B does not include the normal position LV3 described in the first embodiment.

In this way, various modifications are possible to the form of the two-dimensional image IM2C and the three-dimensional image IM3C. The surgery assistance device 10C according to the fourth embodiment is capable of achieving the same effects as those of the first embodiment described above.

### Fifth Embodiment

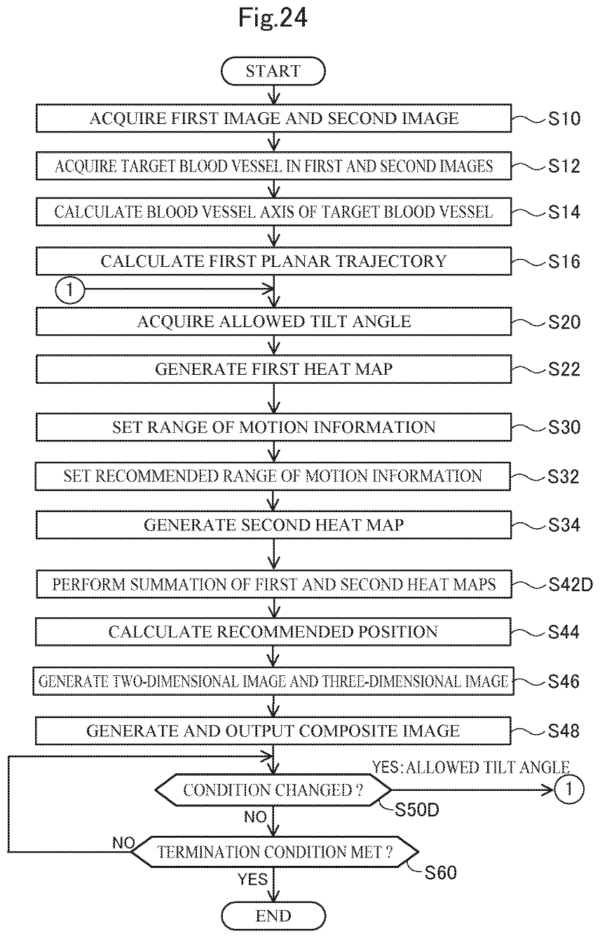

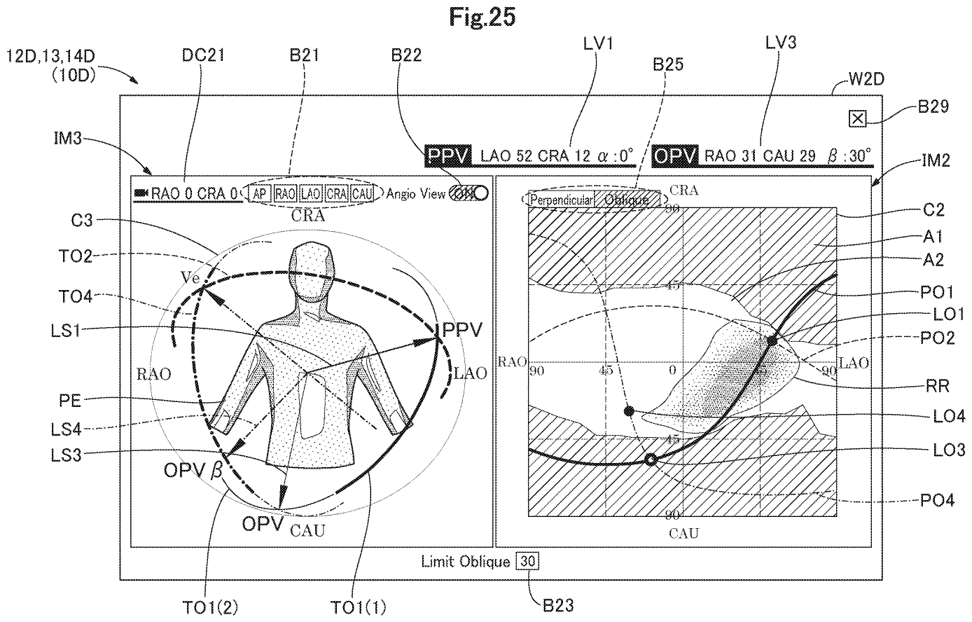

FIG. 24 is a flowchart showing an example of the processing procedure of guide processing according to a fifth embodiment. FIG. 25 is a diagram showing an example of a composite image according to the fifth embodiment. The surgery assistance system 1 according to the fifth embodiment includes a surgery assistance device 10D instead of the surgery assistance device 10 described in the first embodiment. The surgery assistance device 10D includes a two-dimensional image generation unit 12D instead of the two-dimensional image generation unit 12, and a composite image generation unit 14D instead of the composite image generation unit 14.

As shown in FIG. 24, the two-dimensional image generation unit 12D does not execute step S40, in which the weight described in FIG. 6 is acquired. In step S42D, the two-dimensional image generation unit 12D uses a preset weight to perform the summation of the first heat map HM1 and the second heat map HM2. In step S50D, the two-dimensional image generation unit 12D does not perform branching processing according to changes in the weighting conditions. As shown in FIG. 25, the composite image generation unit 14D generates and displays a second screen W2D that does not include a weight specification field B24. The sections of the second screen W2D other than the weight specification field B24 are the same as those described in the first embodiment.

In this way, various modifications are possible to the processing procedure of the guide processing. The two-dimensional image generation unit 12D may not calculate the recommended range RR using a weight specified by the operator, and may calculate the recommended range RR using a predetermined weight. The surgery assistance device 10D according to the fifth embodiment is capable of achieving the same effects as those of the first embodiment described above.

### Sixth Embodiment

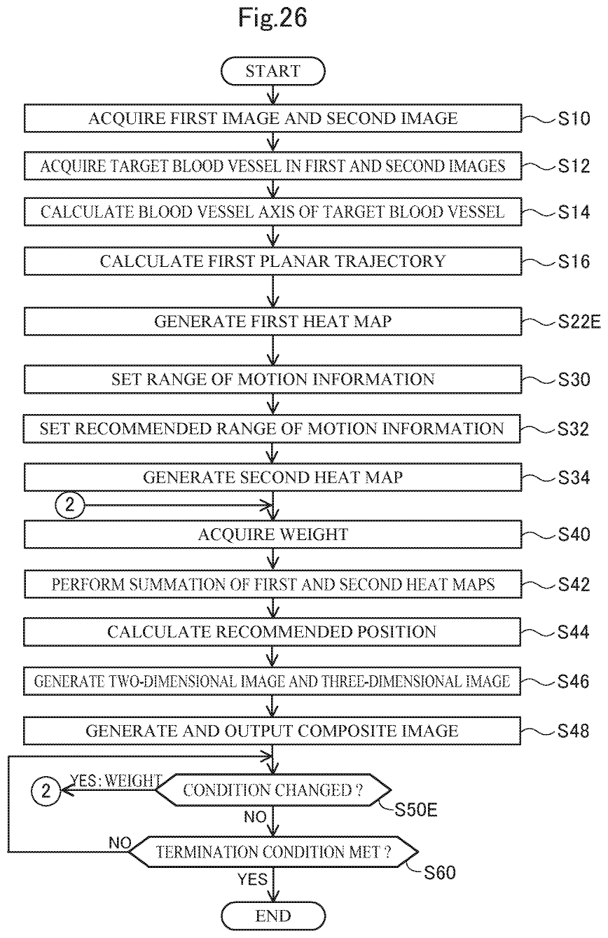

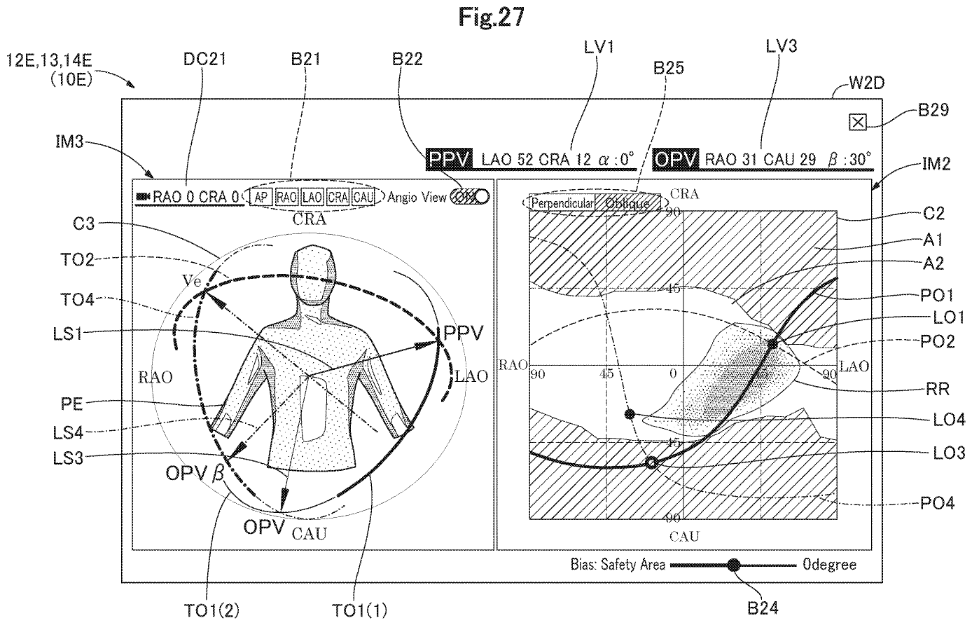

FIG. 26 is a flowchart showing an example of the processing procedure of guide processing according to a sixth embodiment. FIG. 27 is a diagram showing an example of a composite image according to the sixth embodiment. The surgery assistance system 1 according to the sixth embodiment includes a surgery assistance device 10E instead of the surgery assistance device 10 described in the first embodiment. The surgery assistance device 10E includes a two-dimensional image generation unit 12E instead of the two-dimensional image generation unit 12, and a composite image generation unit 14E instead of the composite image generation unit 14.

As shown in FIG. 26, the two-dimensional image generation unit 12E does not execute step S20, in which the allowed tilt angle described in FIG. 6 is acquired. In step S22E, the two-dimensional image generation unit 12E uses a preset allowed tilt angle to generate the first heat map HM1. In step S50E, the two-dimensional image generation unit 12E does not perform branching processing according to changes in the allowed tilt angle. As shown in FIG. 27, the composite image generation unit 14E generates and displays a second screen W2E that does not include the allowed tilt angle specification field B23. The sections of the second screen W2E other than the allowed tilt angle specification field B23 are the same as those described in the first embodiment.

In this way, various modifications are possible to the processing procedure of the guide processing. The two-dimensional image generation unit 12E may not calculate the recommended range RR using an allowed tilt angle specified by the operator, and may calculate the recommended range RR using a predetermined allowed tilt angle. The surgery assistance device 10E according to the sixth embodiment is capable of achieving the same effects as those of the first embodiment described above.

### Seventh Embodiment

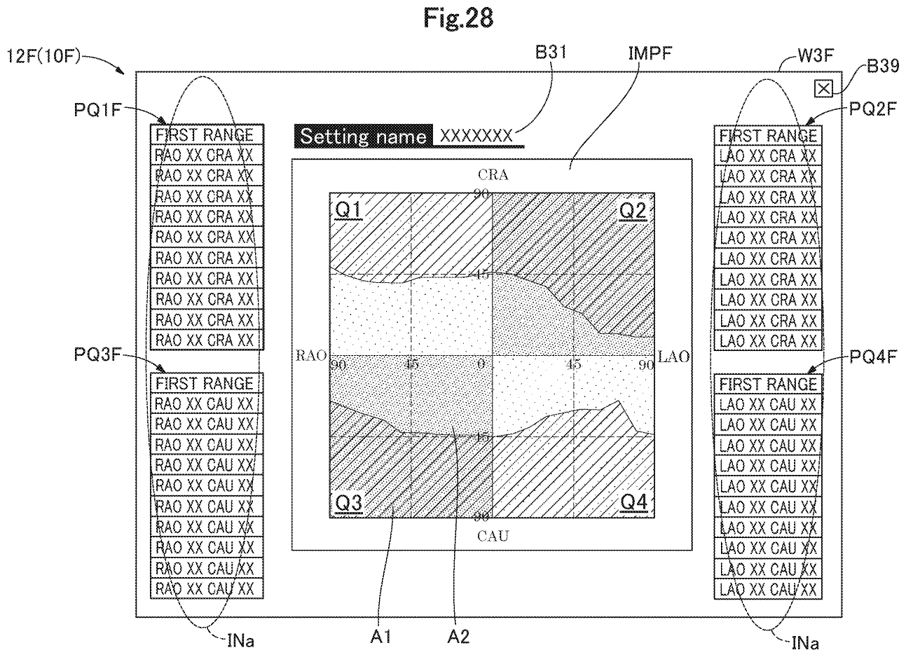

FIG. 28 is a diagram showing an example of a third screen W3F according to a seventh embodiment. The surgery assistance system 1 according to the seventh embodiment includes a surgery assistance device 10F instead of the surgery assistance device 10 described in the first embodiment. The surgery assistance device 10F includes a two-dimensional image generation unit 12F instead of the two-dimensional image generation unit 12.

In step S30 of FIG. 6, the two-dimensional image generation unit 12F acquires the setting of the range of motion information INa by displaying the third screen W3F. The first to fourth range of motion specification fields PQ1F to PQ4F of the third screen W3F are information input fields for setting the first range. The first to fourth range of motion specification fields PQ1F to PQ4F do not include an input field for the second range. In this way, the two-dimensional image generation unit 12F of the present embodiment does not acquire the second range. In the range of motion image IMPF, the non-movable range A1 and the movable range A2 defined by the first to fourth range of motion specification fields PQ1F to PQ4F are drawn on a graph representing the three-dimensional space in which the FPD 21 is placed using a Mercator projection.

After executing step S30 of FIG. 6, the two-dimensional image generation unit 12F omits the execution of step S32. In other words, the two-dimensional image generation unit 12F omits the acquisition of the recommended range of motion information INb. In step S34 of FIG. 6, the two-dimensional image generation unit 12F generates the second heat map HM2 using only the range of motion information Ina acquired in step S30. At this time, the two-dimensional image generation unit 12F, for example, may perform a score adjustment such that a gradation is generated near the curve β1 described in FIGS. 12 and 13. The curve β1 is a boundary line between the non-movable range A1 and the movable range A2.

In this way, various modifications are possible to the method of generating the recommended range RR. The two-dimensional image generation unit 12F may generate the recommended range RR without using the recommended range of motion information INb. The surgery assistance device 10F according to the seventh embodiment is capable of achieving the same effects as those of the first embodiment described above.

### Eighth Embodiment

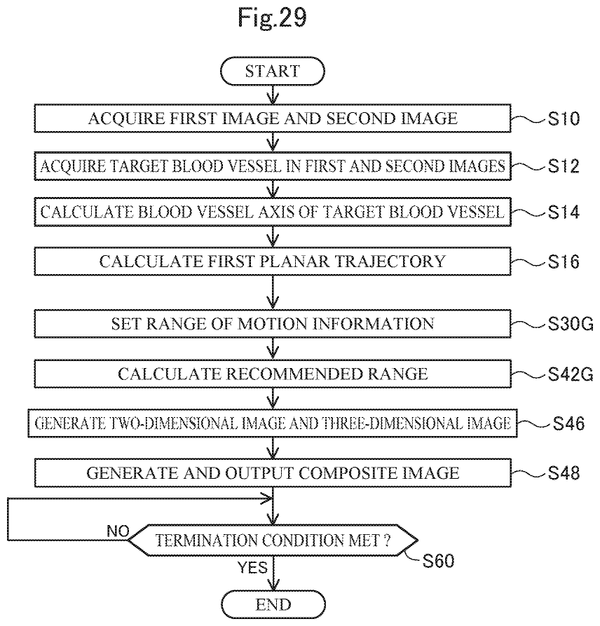

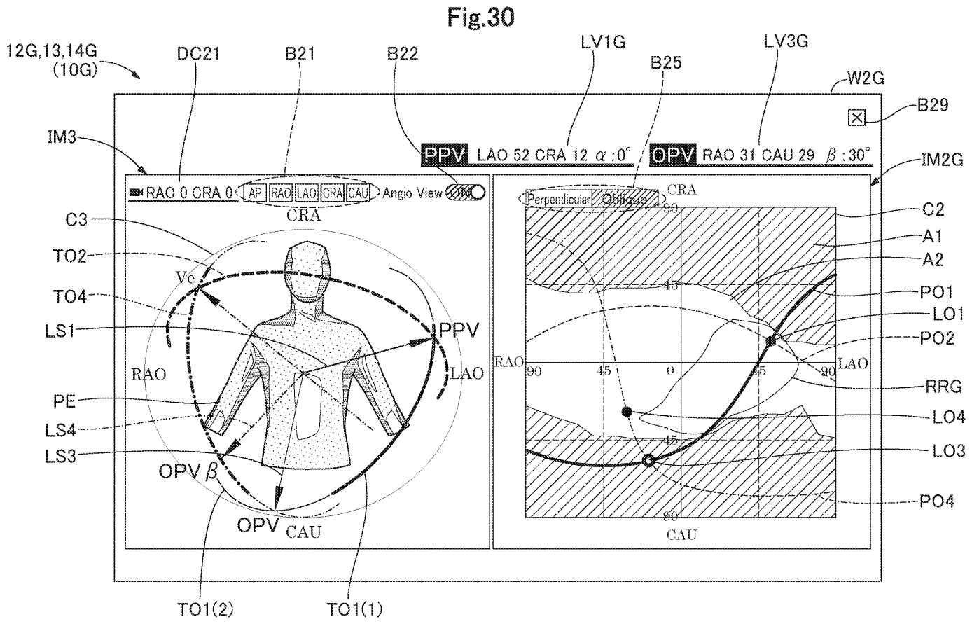

FIG. 29 is a flowchart showing an example of the processing procedure of guide processing according to an eighth embodiment. FIG. 30 is a diagram showing an example of a composite image according to the eighth embodiment. The surgery assistance system 1 according to the eighth embodiment includes a surgery assistance device 10G instead of the surgery assistance device 10 described in the first embodiment. The surgery assistance device 10G includes a two-dimensional image generation unit 12G instead of the two-dimensional image generation unit 12, and a composite image generation unit 14G instead of the composite image generation unit 14. The two-dimensional image generation unit 12G differs from the first embodiment in the processing content of the guide processing.

As shown in FIG. 29, the two-dimensional image generation unit 12G does not execute steps S20 to S22, steps S32 to S34, step S40, step S44, and step S50 described in FIG. 6. The two-dimensional image generation unit 12G executes step S30G instead of step S30 described in FIG. 6, and executes step S42 instead of step S42G. In step S30G, the two-dimensional image generation unit 12G acquires the setting of the range of motion information INa by displaying the third screen W3F described in the seventh embodiment. In step S42G, the two-dimensional image generation unit 12G calculates (derives) a recommended range RRG using only the first planar trajectory PO1 calculated in step S16, and the range of motion information INa acquired in step S30G.

As shown in FIG. 30, the recommended range RRG of the present embodiment is a graphic indicating a range near the first planar trajectory PO1, and inside the movable range A2 derived from the range of motion information INa. The recommended range RRG shown in FIG. 30 is not a heat map such as that of the first embodiment. In other words, the recommended range RRG does not need to have a shading that corresponds to high and low scores. The recommended range RRG may be a heat map similar to that of the first embodiment. The two-dimensional image generation unit 12G generates a two-dimensional image IM2G including such a recommended range RRG. The two-dimensional image generation unit 12G does not calculate the recommended position described in FIG. 19. As shown in FIG. 30, the composite image generation unit 14G generates and displays the second screen W2G. The second screen W2G does not include the allowed tilt angle specification field B23 and the weight specification field B24. The vertical position LV1G and the normal position LV3G of the second screen W2G are not character strings indicating the recommended position.

In this way, various modifications are possible to the method of generating the recommended range RRG. The two-dimensional image generation unit 12G may generate the recommended range RRG without using the recommended range of motion information INb and the allowed tilt angle. The two-dimensional image generation unit 12G may generate the recommended range RRG without using the first heat map HM1 and the second heat map HM2. The surgery assistance device 10G according to the eighth embodiment is capable of achieving the same effects as those of the first embodiment described above.

### Ninth Embodiment

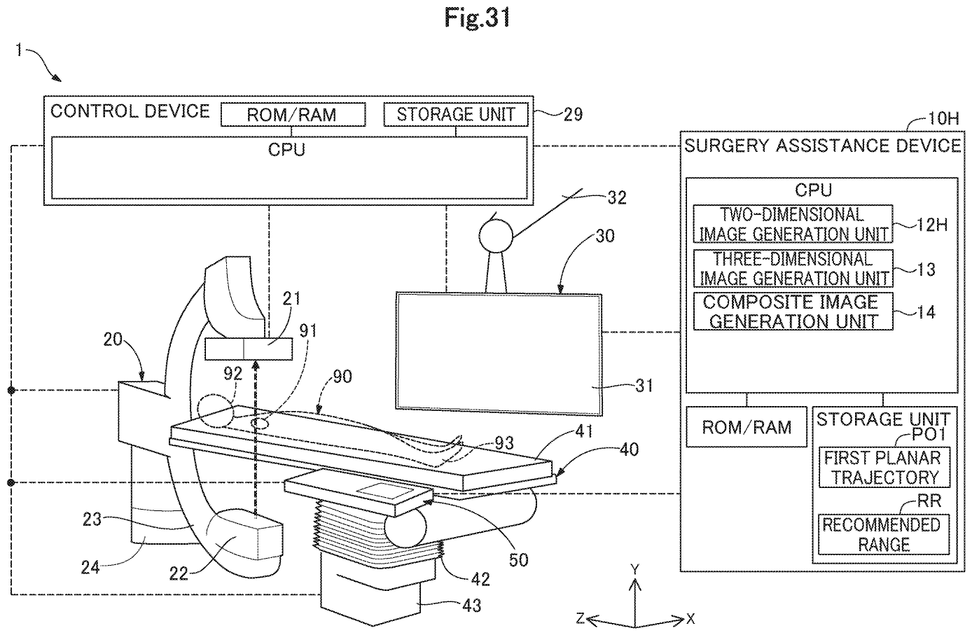

FIG. 31 is an explanatory diagram showing an example of a surgery assistance system 1 according to a ninth embodiment. The surgery assistance system 1 according to the ninth embodiment includes a surgery assistance device 10H instead of the surgery assistance device 10 described in the first embodiment. The surgery assistance device 10H does not include the calculation unit 11, and includes a two-dimensional image generation unit 12H instead of the two-dimensional image generation unit 12. The storage unit of the surgery assistance device 10H stores the first planar trajectory PO1 and the recommended range RR in advance.

The first planar trajectory PO1 is calculated in advance by the same method as in the first embodiment, and is stored in the storage unit. The calculation of the first planar trajectory PO1 may be performed by the surgery assistance device 10H, or by a server or the like that is different from the surgery assistance device 10H. The recommended range RR is generated in advance by the same method as in the first embodiment, and is stored in the storage unit. The generation of the recommended range RR may be performed by the surgery assistance device 10H, or may be performed by a server or the like that is different from the surgery assistance device 10H. The processing procedure of the surgery assistance device 10H is the same as the processing procedure described in FIG. 29, except that step S30G and step S42G are omitted. In addition, the two-dimensional image generation unit 12H may generate the two-dimensional image IM2 in step S46 of FIG. 29 using the first planar trajectory PO1 and the recommended range RR read out from the storage unit.

In this way, various modifications are possible to the processing procedure of the guide processing. The two-dimensional image generation unit 12H may generate the two-dimensional image IM2 using a first planar trajectory PO1 and a recommended range RR that have been prepared in advance. The surgery assistance device 10H according to the ninth embodiment is capable of achieving the same effects as those of the first embodiment described above.

### Tenth Embodiment

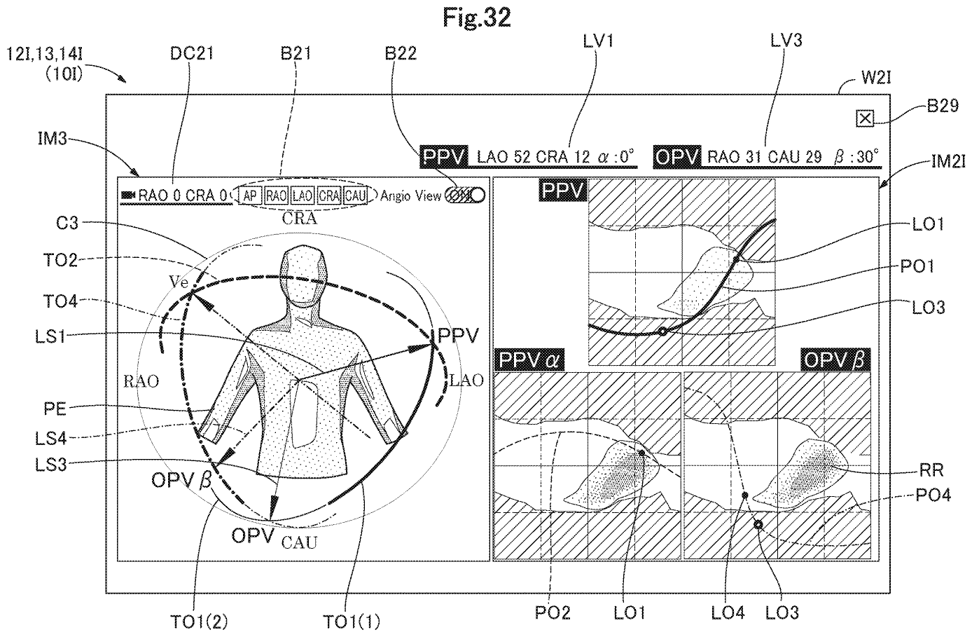

FIG. 32 is a diagram showing an example of a composite image according to a tenth embodiment. The surgery assistance system 1 according to the tenth embodiment includes a surgery assistance device 10I instead of the surgery assistance device 10 described in the first embodiment. The surgery assistance device 10I includes a two-dimensional image generation unit 12I instead of the two-dimensional image generation unit 12, and a composite image generation unit 14I instead of the composite image generation unit 14. The two-dimensional image generation unit 12I generates the two-dimensional image IM2I shown in FIG. 32. The composite image generation unit 14I generates and outputs the second screen W2I shown in FIG. 32.

The two-dimensional image IM2I includes three views, namely a PPV view, a PPVα view, and an OPVβ view. The PPV view includes only the elements described in b1, b2, b3, b4, b7, and b10 in the first embodiment. The PPVα view includes only the elements described in b1, b2, b4, b5, b6, and b10 in the first embodiment. The OPVβ view includes only the elements described in b1, b2, b7, b8, b9, and b10 in the first embodiment. That is, in the tenth embodiment, the PPV view, the PPVα view, and the OPVβ view, which are displayed as a single view in the first embodiment, are each independently and individually displayed.

In this way, various modifications are possible to the form of the two-dimensional image IM2I. The surgery assistance device 10I according to the tenth embodiment is capable of achieving the same effects as those of the first embodiment described above.

### Eleventh Embodiment

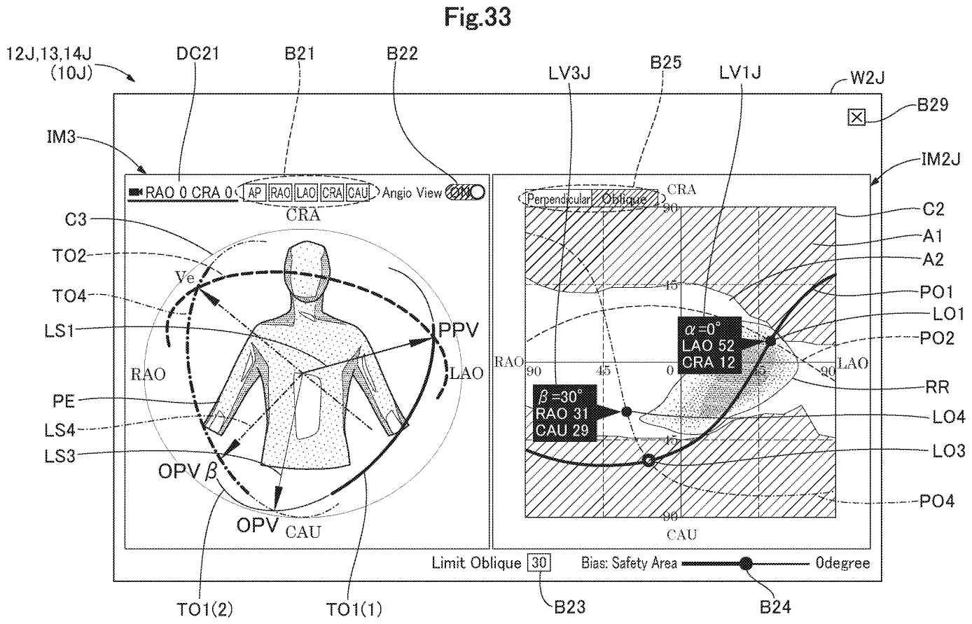

FIG. 33 is a diagram showing an example of a composite image according to an eleventh embodiment. The surgery assistance system 1 according to the eleventh embodiment includes a surgery assistance device 10J instead of the surgery assistance device 10 described in the first embodiment. The surgery assistance device 10J includes a two-dimensional image generation unit 12J instead of the two-dimensional image generation unit 12, and a composite image generation unit 14J instead of the composite image generation unit 14. The two-dimensional image generation unit 12J generates the two-dimensional image IM2J shown in FIG. 33. The composite image generation unit 14J generates and outputs the second screen W2J shown in FIG. 33.

The second screen W2J does not include the vertical position LV1 and the normal position LV3 described in the first embodiment. Instead, the two-dimensional image IM2J includes the vertical position LV1J and the normal position LV3J. The vertical position LV1J is displayed inside a balloon that extends from one of the first position LO1 and the second position LO2, which is represented by a black circle. The normal position LV3J is displayed inside a balloon that extends from one of the third position LO3 and the fourth position LO4, which is represented by a black circle.

In this way, various modifications are possible to the form of the two-dimensional image IM2J. The vertical position LV1J and the normal position LV3J may be included in the two-dimensional image IM2J rather than the composite image. The vertical position LV1J and the normal position LV3J may be included in both the composite image and the two-dimensional image IM2J. The surgery assistance device 10J according to the eleventh embodiment is capable of achieving the same effects as those of the first embodiment described above.

### Modifications of Embodiments

The present disclosure is not limited to the embodiments described above, and may be implemented in various forms without departing from the gist of the present disclosure. For example, the following modifications are also possible.

### First Modification

The forms of the surgery assistance system 1 have been described in the first to eleventh embodiments above. Various modifications are possible to the form of the surgery assistance system 1. For example, in the surgery assistance system 1, the surgery assistance device 10, or 10A to 10J may be connected to the radiation imaging device 20 and the like via the Internet. For example, the display device 30 may be a monitor or touch panel built into the surgery assistance device 10, or 10A to 10J. For example, the radiation imaging device 20 may include a second FPD that is placed in the normal direction of the FPD 21. For example, the surgery assistance system 1 may include another medical device or the like that is not illustrated. Examples of other medical devices include a CT device and an MRI device. In a case where other medical devices are included, the second screen W2 may include images acquired from the other medical devices.

### Second Modification

The forms of the surgery assistance device 10, and 10A to 10J have been described in the first to eleventh embodiments above. Various modifications are possible to the form of the surgery assistance device 10, and 10A to 10J. For example, in the processing procedures described in FIGS. 6, 24, 26, and 29, the execution order of the steps may be changed, at least some of the steps may be omitted, and other steps that have not been described may be executed.

For example, the two-dimensional image generation unit 12 may generate the recommended range RR by taking a hot point into account. A hot point refers to a location that is particularly recommended as the placement position of the FPD 21. For example, a hot point can be the following e1 to e3.

(e1) In a case where a “specific procedure” is performed, a range that is recommended as the placement position of the FPD 21, or a range that is required as the placement position of the FPD 21.
(e2) In a case where a “target blood vessel is in a specific position”, where the blood vessel is the target of the procedure, a range that is recommended as the placement position of the FPD 21, or a range that is required as the placement position of the FPD 21.
(e3) In a case where a “specific operator” performs a procedure, a range that the operator places the FPD 21, or a range that the operator would like to place the FPD 21.

When a hot point is used, the two-dimensional image generation unit 12 has a form in which a hot point can be specified separately from the range of motion information INa and the recommended range of motion information INb in the third screen W3, which is for setting the range of motion information INa and the recommended range of motion information INb. The two-dimensional image generation unit 12 may generate a second heat map HM2 in which the score of the section corresponding to the hot point has been set relatively high, and then calculate the recommended range RR using the second heat map HM2.

For example, the calculation unit 11 may acquire the first image and the second image from a location other than a local folder of the surgery assistance device 10. Examples of other locations include a recording medium built into the radiation imaging device 20, an external device that is directly connected or connected over a network to the radiation imaging device 20, and an external recording medium. For example, the calculation unit 11 may determine the blood vessel axis vector by a different method to the method described in step S14. For example, instead of determining the blood vessel axis vector, the calculation unit 11 may calculate the blood vessel axis Ve by obtaining the start coordinates and end coordinates of the target blood vessel. For example, step S50 may be omitted. In this case, after completing step S48, the processing may directly transition to step S60.

For example, the first screen W1 is merely an example, and various modifications are possible. For example, the layout of each item on the screen, the presence or absence of the items, and the operation method of the screen can be changed as appropriate. For example, the second vector drawing button B123, the display panel DC12, and the display panel DC14 may be omitted. For example, the trajectory display button B11 may be omitted, and the processing may automatically proceed to the following step once vector drawing is complete. For example, at least one or more of the cancel button B121, the magnification button B124, the range movement button B125, and the redo button B126 may be omitted. For example, the first screen W1 may acquire touch operations made by a finger instead of mouse operations, or together with mouse operations.

For example, the second screen W2 is merely an example, and various modifications are possible. For example, the layout of each item on the screen, the presence or absence of the items, and the operation method of the screen can be changed as appropriate. For example, the two-dimensional image IM2 does not have to include the non-movable range A1 and the movable range A2. For example, the two-dimensional image IM2 does not have to include the mode specification button B25. For example, the two-dimensional image IM2 may include other items that have not been described above. For example, the three-dimensional image IM3 does not have to include the patient image PE. For example, the three-dimensional image IM3 does not have to include at least one or more of the display panel DC21, the viewpoint change buttons B21, and the head portion position fixing button B22. For example, the three-dimensional image IM3 may include other items that have not been described above. For example, in a case where the viewpoint change buttons B21 and the head portion position fixing button B22 are omitted, the viewpoint of the three-dimensional image IM3 may be fixed. For example, the second screen W2 may acquire touch operations made by a finger instead of mouse operations, or together with mouse operations.

In the embodiment described above, it has been assumed that, in the second screen W2, the blood vessel axis Ve passes through the origin of the three-dimensional coordinates C3. However, the origins of the blood vessel axis Ve and three-dimensional coordinates C3 do not necessarily have to coincide. Specifically, if the deviation between the blood vessel axis Ve and the origin of the three-dimensional coordinates C3 is within a predetermined error range, it can be considered that “the blood vessel axis Ve passes through the origin of the three-dimensional coordinates C3”.

In the embodiment described above, the first to fourth line segments LS1 to LS4 in the three-dimensional image IM3 are changed by operating the first to fourth positions LO1 to LO4 of the two-dimensional image IM2. In contrast, by operating the first to fourth line segments LS1 to LS4 of the three-dimensional image IM3, the first to fourth positions LO1 to LO4 of the two-dimensional image IM2 may be changed so as to follow the change in the positions of the first to fourth line segments LS1 to LS4. By operating one of the positions in the two-dimensional image IM2 and the line segments in the three-dimensional image IM3, the other can be made to follow.

### Third Modification

The forms of the surgery assistance devices 10, and 10A to 10J of the first to eleventh embodiments described above, and the aspects of the first and second modifications described above may be combined as appropriate. For example, the elements described in the second, third, tenth, and eleventh embodiments may be combined to form a new second screen W2. For example, it may be possible to select the display mode of the second screen W2 from among the display modes of the second, third, tenth, and eleventh embodiments. In this case, for example, a mode is possible in which a setting is made using a setting screen, or a mode is possible in which a selection is made at the start of the guide processing. For example, in any of the second to eighth embodiments, and in the tenth and eleventh embodiments, the “mode that prepares the recommended range RR in advance” described in the second embodiment may be adopted.

The present aspect has been described above based on the embodiments and the modifications. The embodiments of the aspect described above are intended to facilitate understanding of the present aspect, and do not limit the present aspect. The present aspect may be modified and improved without departing from the gist and the scope of the claims, and the present aspect includes equivalents thereof. If the technical features are not described as essential in the present specification, the technical features may be appropriately removed.

What is claimed is:

1. A surgery assistance device comprising
circuitry configured to:
generate an image showing a placement position of a flat panel detector (FPD) when a target blood vessel is imaged using the FPD, the FPD being configured to be moved along a surface of a virtual sphere having a predetermined radius and being centered at an origin of a three-dimensional space,
acquire range of motion information that defines a first range in which the FPD is configured to be moved, and
generate a two-dimensional image showing the placement position of the FPD in two-dimensional coordinates, and which is a two-dimensional image including a first planar trajectory on which the placement position of the FPD is located in a first direction, which is perpendicular to an extending direction of the target blood vessel, and a recommended range showing a range derived using the range of motion information and the first planar trajectory, which indicates a range that is recommended as the placement position of the FPD.

2. The surgery assistance device according to claim 1,
wherein:
the circuitry is further configured to derive the recommended range using the range of motion information and the first planar trajectory.

3. The surgery assistance device according to claim 2,
wherein:
the circuitry is further configured to:
acquire recommended range of motion information that defines a second range within the first range wherein within the second rangethe FPD can be moved more easily than within at least a portion of the first range, and
derive the recommended range using the recommended range of motion information in addition to the range of motion information and first planar trajectory,
and wherein, in the recommended range, a visibility of a section corresponding to the second range is made higher than a visibility of a section corresponding to only the first range.

4. The surgery assistance device according to claim 3,
wherein:
the recommended range of motion information includes first recommended range of motion information and second recommended range of motion information, and
the circuitry is configured to derive the recommended range using one of the first recommended range of motion information and the second recommended range of motion information that has been specified by an operator.

5. The surgery assistance device according to claim 3,
wherein:
the circuitry is further configured to:
acquire an allowed tilt angle, being an allowed tilt angle of the FPD, that represents an upper limit value indicating up to how many degrees the FPD is configured to be tilted when placed in an arbitrary placement position,
derive the recommended range using the allowed tilt angle in addition to the range of motion information, the first planar trajectory, and the recommended range of motion information,
in the recommended range, cause a visibility of a section far from the upper limit value to be higher than a visibility of a section near the upper limit value, and
derive the recommended range by combining a first map that increases or decreases a visibility based on the first planar trajectory and the allowed tilt angle, and a second map that increases or decreases a visibility based on the range of motion information and the recommended range of motion information.

6. The surgery assistance device according to claim 5,
wherein:
the circuitry is configured to acquire an angle set by an operator as the allowed tilt angle, and is configured to then derive the recommended range.

7. The surgery assistance device according to claim 5,
wherein:
the circuitry is configured to derive the recommended range by weighting and combining the first map and the second map.

8. The surgery assistance device according to claim 7,
wherein:
the circuitry is configured to derive the recommended range by acquiring a weight that has been set by an operator, and is configured to then combine the first map and the second map using the weight.

9. The surgery assistance device according to claim 5,
wherein:
the circuitry is configured to generate the two-dimensional image further including, with respect to each of (i) a first position, which is an arbitrary placement position of the FPD on the first planar trajectory, (ii) a second position, which is an arbitrary placement position of the FPD on a second planar trajectory, the second planar trajectory being a trajectory that causes the placement position of the FPD to be positioned in a second direction, which is on a first virtual plane passing through the extending direction of the target blood vessel and a virtual line segment extending from the origin of the three-dimensional space to the first position, (iii) a third position, which is in a position away from the first position on the first planar trajectory, and is on a second virtual plane obtained by rotating the first virtual plane by 90° around the extending direction of the target blood vessel, and (iv) a fourth position, which is an arbitrary placement position of the FPD on a fourth planar trajectory, the fourth planar trajectory being a trajectory that causes the placement position of the FPD to be positioned in a fourth direction on the second virtual plane,
a first character string indicating a position of the FPD for placing the FPD in either one of the first position and the second position, and
a second character string indicating a position of the FPD for placing the FPD in either one of the third position and the fourth position.

10. The surgery assistance device according to claim 9,
wherein:
the circuitry is configured to generate a plurality of candidates of the first to fourth positions, in which either one of the first position and the second position are included in the recommended range, and either one of the third position and the fourth position are included in the recommended range, and
wherein one candidate selected from the plurality of candidates that results in a smallest sum of a tilt angle of the FPD indicated by the second position and a tilt angle of the FPD indicated by the fourth position is used to determine the first character string and the second character string.

11. A method, executed by a computer, of assisting a surgery that includes a process of capturing a target blood vessel using a flat panel detector (FPD), the FPD being configured to be moved along a surface of a virtual sphere having a predetermined radius and being centered at an origin of a three-dimensional space, the method comprising:
acquiring range of motion information that defines a first range in which the FPD is configured to be moved, and
generating a two-dimensional image that shows a placement position of the FPD in two-dimensional coordinates, and which includes a first planar trajectory on which the placement position of the FPD is located in a first direction, which is perpendicular to an extending direction of the target blood vessel, and a recommended range showing a range derived using the range of motion information and the first planar trajectory, which indicates a range that is recommended as the placement position of the FPD.

12. A non-transitory computer-readable medium storing a computer program that assists a surgery that includes a process of capturing, a target blood vessel using a flat panel detector (FPD), the FPD being configured to be moved along a surface of a virtual sphere having a predetermined radius and being centered at an origin of a three-dimensional space, and, when executed, the program is configured to cause a computer to:
acquire range of motion information that defines a first range in which the FPD is configured to be moved, and
generate a two-dimensional image that shows the placement position of the FPD in two-dimensional coordinates, and which includes a first planar trajectory on which the placement position of the FPD is located in a first direction, which is perpendicular to an extending direction of the target blood vessel, and a recommended range showing a range derived using the range of motion information and the first planar trajectory, which indicates a range that is recommended as the placement position of the FPD.

13. The non-transitory computer-readable medium according to claim 12,
wherein:
the recommended range is derived using the range of motion information and the first planar trajectory.

14. The non-transitory computer-readable medium according to claim 13,
wherein:
recommended range of motion information is acquired that defines a second range within the first range wherein within the second rangethe FPD can be moved more easily than within at least a portion of the first range,
the recommended range is derived using the recommended range of motion information in addition to the range of motion information and first planar trajectory, and
in the recommended range, a visibility of a section corresponding to the second range is made higher than a visibility of a section corresponding to only the first range.

15. The non-transitory computer-readable medium according to claim 14,
wherein:
the recommended range of motion information includes first recommended range of motion information and second recommended range of motion information, and
the recommended range is derived using one of the first recommended range of motion information and the second recommended range of motion information that has been specified by an operator.

16. The non-transitory computer-readable medium according to claim 14,
wherein:
the program is further configured to cause the computer to acquire an allowed tilt angle, the allowed tilt angle being an allowed tilt angle of the FPD, that represents an upper limit value indicating up to how many degrees the FPD is configured to be tilted when placed in an arbitrary placement position,
and wherein the recommended range is derived using the allowed tilt angle in addition to the range of motion information, the first planar trajectory, and the recommended range of motion information,
in the recommended range, a visibility of a section far from the upper limit value is caused to be higher than a visibility of a section near the upper limit value, and
the recommended range is derived by combining a first map that increases or decreases a visibility based on the first planar trajectory and the allowed tilt angle, and a second map that increases or decreases a visibility based on the range of motion information and the recommended range of motion information.

17. The non-transitory computer-readable medium according to claim 16,
wherein:
an angle set by an operator is acquired as the allowed tilt angle, and the recommended range is derived.

18. The non-transitory computer-readable medium according to claim 16,
wherein:
the recommended range is derived by weighting and combining the first map and the second map.

19. The non-transitory computer-readable medium according to claim 18,
wherein:
the recommended range is derived by acquiring a weight that has been set by an operator, and then the first map and the second map are combined using the weight.

20. The non-transitory computer-readable medium according to claim 16,
wherein:
the two-dimensional image is generated further including, with respect to each of (i) a first position, which is an arbitrary placement position of the FPD on the first planar trajectory, (ii) a second position, which is an arbitrary placement position of the FPD on a second planar trajectory, the second planar trajectory being a trajectory that causes the placement position of the FPD to be positioned in a second direction, which is on a first virtual plane passing through the extending direction of the target blood vessel and a virtual line segment extending from the origin of the three-dimensional space to the first position, (iii) a third position, which is in a position away from the first position on the first planar trajectory, and is on a second virtual plane obtained by rotating the first virtual plane by 90° around the extending direction of the target blood vessel, and (iv) a fourth position, which is an arbitrary placement position of the FPD on a fourth planar trajectory, the fourth planar trajectory being a trajectory that causes the placement position of the FPD to be positioned in a fourth direction on the second virtual plane,
a first character string indicating a position of the FPD for placing the FPD in either one of the first position and the second position, and
a second character string indicating a position of the FPD for placing the FPD in either one of the third position and the fourth position.
